(19) United States
(12) Patent Application Publication
(10) Pub. No.: US 2026/0124203 A1
(43) Pub. Date: May 7, 2026

(54) CHEMICAL SYNTHETIC LETHALITY FOR ANTIMICROBIAL THERAPY

(71) Applicants: The Trustees of Princeton University, Princeton, NJ (US); EMORY UNIVERSITY, Atlanta, GA (US); University of Kansas, Lawrence, KS (US)

(72) Inventors: Mohammad Seyedsayamdost, Princeton, NJ (US); Yifan Zhang, Princeton, NJ (US); Josephine Chandler, Lawrence, KS (US); Jennifer Klaus, Lawrence, KS (US); Katherine Davis, Atlanta, GA (US); Kirklin McWhorter, Atlanta, GA (US); Paul Rosen, Princeton, NJ (US)

(73) Assignees: The Trustees of Princeton University, Princeton, NJ (US); EMORY UNIVERSITY, Atlanta, GA (US); University of Kansas, Lawrence, KS (US)

(21) Appl. No.: 19/380,619

(22) Filed: Nov. 5, 2025

Related U.S. Application Data
(60) Provisional application No. 63/716,570, filed on Nov. 5, 2024.

(51) Int. Cl. A61K 31/505; A61K 31/352; A61K 31/365; A61P 31/04; C12Q 1/20

(52) U.S. Cl. CPC A61K 31/505; A61K 31/352; A61K 31/365; A61P 31/04; C12Q 1/20

(57) ABSTRACT

The present disclosure provides a system for treating melioidosis, comprising a first antimicrobial agent comprising trimethoprim at a subinhibitory concentration and a second antimicrobial agent comprising a FolE2 enzyme inhibitor selected from dehydrocostus lactone, parthenolide, or β-lapachone, wherein the combination of the first antimicrobial agent and the second antimicrobial agent exhibits chemical synthetic lethality against Burkholderia pseudomallei while having minimal effect on commensal bacteria. The subinhibitory concentration of trimethoprim ranges between 5 μM and 30 μM. The FolE2 enzyme inhibitor acts as a mechanism-based inhibitor that covalently modifies a catalytic cysteine residue (Cys154) of the FolE2 enzyme. The combination exhibits greater than 90% growth suppression against Burkholderia pseudomallei. Methods for treating melioidosis and identifying antimicrobial drug targets using chemical synthetic lethality screening approaches are also provided.

### CROSS-REFERENCE TO RELATED APPLICATIONS

This application claims priority to U.S. Provisional Application No. 63/716,570, titled Combatting Melioidosis with Chemical Synthetic Lethality, filed Nov. 5, 2024, which is hereby incorporated by reference in its entirety.

### STATEMENT REGARDING FEDERALLY SPONSORED RESEARCH OR DEVELOPMENT

This invention was made with government support under Grant Nos. GM133572, GM147557, and GM140034 awarded by the National Institutes of Health and DE-AC02-06CH11357 awarded by the Department of Energy. The government has certain rights in the invention.

### TECHNICAL FIELD

The present disclosure relates to antimicrobial therapy methods, and more particularly to chemical synthetic lethality approaches for treating bacterial infections by combining subinhibitory concentrations of known antibiotics with enzyme inhibitors to selectively target pathogenic bacteria while sparing commensal microorganisms.

### BACKGROUND

Antimicrobial resistance represents a growing global health challenge that threatens the effectiveness of existing antibiotic therapies. The emergence of resistant bacterial strains has outpaced the development of new antimicrobial agents, creating an urgent need for novel therapeutic approaches. Traditional antibiotic discovery methods have increasingly yielded diminishing returns, with many pharmaceutical companies reducing their investment in antimicrobial research due to economic and scientific challenges.

Conventional antimicrobial therapy typically relies on single-agent treatments that target widely distributed bacterial pathways or structures. While this approach has proven effective against many pathogens, it often results in broad-spectrum activity that can disrupt beneficial microbial communities, particularly in the gastrointestinal tract. Such disruption of the microbiome can lead to secondary infections, antibiotic-associated diarrhea, and other adverse effects that complicate patient care.

The concept of synthetic lethality, originally developed in genetics, describes a phenomenon where the simultaneous disruption of two genes results in cell death, while disruption of either gene alone is tolerated. This principle has been successfully applied in cancer therapy, where combinations of targeted agents can selectively kill cancer cells while sparing normal cells. However, the application of synthetic lethality principles to antimicrobial therapy remains largely unexplored.

Many bacterial pathogens possess large genomes that encode extensive metabolic capabilities and redundant pathways, making them particularly challenging to treat with conventional single-agent therapies. These organisms can often compensate for the inhibition of one pathway by upregulating alternative metabolic routes, leading to treatment failure or the development of resistance. The metabolic flexibility of such pathogens suggests that combination approaches targeting multiple pathways simultaneously may be more effective.

Current combination antimicrobial therapies, such as trimethoprim-sulfamethoxazole, typically employ two agents at therapeutic concentrations to achieve synergistic effects or prevent resistance development. However, these combinations often retain the broad-spectrum activity of their individual components, continuing to impact commensal bacteria and potentially causing collateral damage to the host microbiome.

The identification of conditionally essential genes and pathways in bacterial pathogens offers new opportunities for selective antimicrobial targeting. These pathways may be dispensable under normal growth conditions but become essential when bacteria are subjected to specific stresses or metabolic perturbations. Such conditional essentiality could provide a basis for developing more selective antimicrobial strategies that exploit the unique metabolic requirements of pathogens under stress conditions.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a system for treating melioidosis is provided. The system comprises a first antimicrobial agent comprising trimethoprim at a subinhibitory concentration and a second antimicrobial agent comprising a FolE2 enzyme inhibitor selected from dehydrocostus lactone, parthenolide, or β-lapachone. The combination of the first antimicrobial agent and the second antimicrobial agent exhibits chemical synthetic lethality against Burkholderia pseudomallei while having minimal effect on commensal bacteria. This combination approach provides selective antimicrobial activity by exploiting the metabolic vulnerabilities of pathogenic bacteria under stress conditions while preserving beneficial microorganisms. The chemical synthetic lethality mechanism allows for targeted therapy that reduces collateral damage to the host microbiome compared to conventional broad-spectrum antibiotics.

According to other aspects of the present disclosure, the system may include one or more of the following features. The subinhibitory concentration of trimethoprim may be between 5 μM and 30 μM. This concentration range maintains the metabolic stress on the pathogen without causing direct growth inhibition, thereby creating the conditional essentiality for the FolE2 enzyme while minimizing toxicity to the host and commensal organisms.

According to other aspects of the present disclosure, the subinhibitory concentration of trimethoprim may be between 5 μM and 30 μM. This refined concentration range provides optimal conditions for inducing the secondary metabolite stress response in Burkholderia species, leading to upregulation of folE2 expression and creating the metabolic dependency that enables the synthetic lethal interaction.

According to other aspects of the present disclosure, the FolE2 enzyme inhibitor may be dehydrocostus lactone. Dehydrocostus lactone offers potent and selective inhibition of the FolE2 enzyme through its mechanism-based inhibition properties, providing effective antimicrobial activity when combined with subinhibitory trimethoprim concentrations.

According to other aspects of the present disclosure, the dehydrocostus lactone may have an IC₅₀ value of between 1.7 μM and 2.5 μM in the presence of trimethoprim. This potency range demonstrates the enhanced effectiveness of dehydrocostus lactone under conditions of folate stress, confirming the synthetic lethal interaction and providing a therapeutic window for effective treatment.

According to other aspects of the present disclosure, the FolE2 enzyme inhibitor may be β-lapachone.

β-lapachone provides an alternative mechanism-based inhibitor option with demonstrated efficacy against the FolE2 enzyme, offering flexibility in treatment approaches and potential for addressing resistance development.

According to other aspects of the present disclosure, the β-lapachone may have an IC₅₀ value of between 3.2 μM and 6.4 μM in the presence of trimethoprim. This potency profile for β-lapachone demonstrates its effectiveness as a FolE2 inhibitor in the synthetic lethal combination, providing another viable therapeutic option with measurable antimicrobial activity.

According to other aspects of the present disclosure, the FolE2 enzyme inhibitor may be parthenolide. Parthenolide represents another sesquiterpene lactone option for FolE2 inhibition, expanding the available therapeutic compounds and providing structural diversity within the inhibitor class.

According to other aspects of the present disclosure, the combination may exhibit greater than 90% growth suppression against Burkholderia pseudomallei. This high level of growth suppression demonstrates the potent antimicrobial efficacy of the synthetic lethal combination, indicating its potential as an effective therapeutic approach for treating melioidosis infections.

According to other aspects of the present disclosure, the FolE2 enzyme inhibitor may act as a mechanism-based inhibitor that covalently modifies a catalytic cysteine residue of the FolE2 enzyme. The mechanism-based inhibition provides irreversible enzyme inactivation, leading to sustained antimicrobial effects and reducing the likelihood of rapid resistance development through target modification.

According to other aspects of the present disclosure, the catalytic cysteine residue may be Cys154. The specific targeting of Cys154 provides a defined molecular mechanism for enzyme inhibition, enabling rational drug design approaches and facilitating the development of additional inhibitors with similar mechanisms of action.

According to another aspect of the present disclosure, a method for treating melioidosis in a subject is provided. The method comprises administering to the subject a therapeutically effective amount of a pharmaceutical composition comprising trimethoprim at a subinhibitory concentration and a FolE2 enzyme inhibitor. The FolE2 enzyme inhibitor is selected from dehydrocostus lactone, parthenolide, or β-lapachone. The combination exhibits chemical synthetic lethality against Burkholderia pseudomallei. This therapeutic method provides a novel treatment approach for melioidosis that leverages chemical synthetic lethality to achieve selective antimicrobial activity, potentially improving patient outcomes while reducing adverse effects associated with broad-spectrum antibiotic therapy.

According to other aspects of the present disclosure, the method may include one or more of the following features. The subinhibitory concentration of trimethoprim may be between 5 μM and 30 μM, such as about 20 μM. This dosing range provides therapeutic efficacy while maintaining the subinhibitory nature of the trimethoprim component, enabling the synthetic lethal interaction without causing direct antimicrobial effects from trimethoprim alone.

According to other aspects of the present disclosure, the FolE2 enzyme inhibitor may be dehydrocostus lactone having an IC₅₀ value of between 1.7 μM and 2.5 μM in the presence of trimethoprim. This specific inhibitor and potency range provides a defined therapeutic target with demonstrated efficacy, enabling precise dosing strategies and predictable antimicrobial outcomes in clinical applications.

According to other aspects of the present disclosure, the FolE2 enzyme inhibitor may act as a mechanism-based inhibitor that covalently modifies a catalytic cysteine residue of the FolE2 enzyme. The mechanism-based inhibition ensures sustained enzyme inactivation, providing prolonged antimicrobial effects and potentially reducing dosing frequency requirements for patient treatment regimens.

According to other aspects of the present disclosure, the catalytic cysteine residue may be Cys154. The specific targeting of Cys154 provides a well-defined molecular target for therapeutic intervention, enabling structure-based drug design approaches and facilitating the development of next-generation inhibitors with improved properties.

According to other aspects of the present disclosure, the combination may exhibit greater than 90% growth suppression against Burkholderia pseudomallei while having minimal effect on commensal bacteria selected from Bacteroides fragilis, Bifidobacterium longum, Clostridium sporogenes, and Parabacteroides distasonis. This selective antimicrobial profile demonstrates the advantage of the synthetic lethal approach in preserving beneficial microorganisms while effectively targeting the pathogen, potentially reducing microbiome disruption and associated complications in treated patients.

According to another aspect of the present disclosure, a method for identifying antimicrobial drug targets is provided. The method comprises screening a library of compounds for growth inhibition of a target bacterial pathogen in the presence of a subinhibitory concentration of a known antibiotic, identifying compounds that exhibit no growth inhibition individually but cause growth inhibition when combined with the subinhibitory concentration of the known antibiotic, determining the molecular target of the identified compounds, and validating the molecular target as a conditionally essential enzyme that becomes essential in the presence of the subinhibitory concentration of the known antibiotic. This screening methodology provides a systematic approach for discovering new antimicrobial targets and therapeutic combinations, expanding the pipeline of potential treatments and enabling the identification of previously unexploited vulnerabilities in bacterial pathogens.

According to other aspects of the present disclosure, the method may include one or more of the following features. The library of compounds may comprise natural products and FDA-approved small-molecule drugs. The use of established compound libraries accelerates the drug discovery process by leveraging existing safety and pharmacological data, potentially reducing development timelines and regulatory requirements for identified therapeutic combinations.

According to other aspects of the present disclosure, growth inhibition may be measured by optical density at 600 nm after incubation for 24 to 48 hours. This standardized measurement approach provides reproducible and quantitative assessment of antimicrobial activity, enabling reliable comparison of compound efficacy and facilitating the identification of promising therapeutic candidates.

According to other aspects of the present disclosure, the identified compounds may be mechanism-based inhibitors that covalently modify a catalytic amino acid residue. The identification of mechanism-based inhibitors provides compounds with sustained antimicrobial activity and reduced potential for rapid resistance development, offering advantages over reversible inhibitors in therapeutic applications.

According to other aspects of the present disclosure, the mechanism-based inhibitors may comprise sesquiterpene lactones with exocyclic enone moieties. This structural class provides a defined chemical framework for further drug development efforts, enabling medicinal chemistry optimization to improve potency, selectivity, and pharmacological properties of the inhibitors.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1A depicts an abbreviated one-carbon metabolic pathway focusing on steps catalyzed by FolE, FolE2, FolP, and FolA. The latter two are inhibited by SMX and TMP, respectively.

FIG. 1B is a plot showing transcriptional and translational changes of one-carbon metabolite genes in response to low-dose TMP. FolE2 is transcriptionally the most induced gene and translationally the second-most induced protein in the presence of low-dose TMP. Bars represent standard error from three biological replicates.

FIG. 1C is a graph showing growth curves of wt B. thailandensis vehicle control, wt B. thailandensis with 20 μM TMP, ΔfolE2 vehicle control, and ΔfolE2 with 20 μM TMP. Bars represent standard error from three biological replicates.

FIG. 2 illustrates, at the top, a high-throughput screen with 1320 compounds monitoring wt B. thailandensis growth in the absence (squares) or presence (circles) of low-dose TMP. Hits are those that reduce growth by 2 sigma or more in the presence of TMP but do not do so in its absence, and at the bottom, structures of 10 inhibitors that satisfy hit criteria and validated independently in two assay formats (96-well plates and flask culture) and are commercially available.

FIG. 3A is a plot showing growth curves of wt B. thailandensis in the presence of vehicle control, low-dose TMP, DHL, and low-dose TMP and DHL. Also shown are traces for ΔfolE2 vehicle control and ΔfolE2 with low-dose TMP. Error bars represent standard error from three biological replicates.

FIG. 3B is as chart showing quantification of endpoint OD₆₀₀ (at 48 h) from FIG. 3A. The combination of DHL and low-dose TMP results in a 20-fold lower OD₆₀₀. Error bars represent standard error from three biological replicates.

FIG. 3C is a plot showing half-maximal inhibitory concentration of DHL of wt B. thailandensis in the presence of 0 (triangle), 5 (diamond), 10 (square), and 20 (circle) M TMP. Error bars represent standard error from three biological replicates.

FIG. 3D is a chart showing quantification of IC₅₀ from the data of FIG. 3C. Error bars represent standard error from three biological replicates.

FIG. 4A is an illustration showing a reaction carried out by FolE2 (and FolE).

FIG. 4B is a graph showing Michaelis-Menten curve for FolE2 yields k_{cat} and Kₘ of 74 min⁻¹ and 22 μM.

FIGS. 4C-4E are graphs showing inhibition of FolE2 by DHL (4C), parthenolide (4D), and β-lapachone (4E). Shown are inhibition curves at substrate Kₘ yielding apparent Kᵢ values of 87±21, 21.9±4.0, and 2.9±0.5.

FIG. 5A illustrates an X-ray crystal structure of B. pseudomallei FolE2 bound to the covalent inhibitor DHL, providing an overview of the apo FolE2 homotetramer solved to 2.02 Å.

FIG. 5B illustrates metal-binding site of the as-purified enzyme.

FIG. 5C illustrates structure of the DHL-inhibited enzyme in which the metal-coordinating Cys154 is covalently linked to DHL. Locations of possible additional crosslinks are denoted with asterisks.

FIG. 5D illustrates Cys154 reacting with the exo enone of DHL to yield an inhibited complex, which is observed crystallographically.

FIG. 6 illustrates a heatmap showing specificity of synthetic lethal combinations. TMP/DHL, TMP/BL, and TMP/parthenolide form synthetic lethal combinations that are highly selective against B. thailandensis. Among several commensal strains, only E. coli is inhibited. Note that other common antibiotics are not only toxic toward B. thailandensis but also cause collateral damage.

FIG. 7 is a table showing Half-maximal inhibitory concentration and minimal inhibitory concentration of inhibitors identified against B. thailandensis and B. pseudomallei in the presence of TMP concentrations indicated as well as Ki values for B. pseudomallei FolE2 inhibitors. The TMP concentrations below have no effect on B. thailandensis growth when used in isolation. The MIC of TMP alone on B. thailandensis and B. pseudomallei is 84±14 and 120 μM, respectively.

FIGS. 8A and 8B are bar graphs showing complementation of folE2, via plasmid-based expression, in the ΔfolE2 mutant rescues the majority of its growth (8A); however, thymine does not rescue growth of the ΔfolE2 mutant with TMP or the wt with TMP/SMX (8B). Analogous results (to those with thymine) were obtained with thymidine.

FIG. 9 illustrates chemical structures of various pharmaceutical compounds and natural products, and specifically compounds identified in the initial screen that did not validate because they did not inhibit B. thailandensis growth in both growth formats (i.e. flask and 96-well growth) when combined with TMP, or exhibited toxicity individually in one of the two formats.

FIG. 10 is a graph showing minimum inhibitory concentrations (MICs) of clinically used antibiotics against B. thailandensis. The concentrations used were 10 μM for ceftazidime, 6 μM for meropenem, 10 μM for doxycycline, and 20 μM for the TMP component of TMP/SMX. Maintaining a 1:20 mass ratio of TMP/SMX results in a SMX concentration of 458 μM.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A system for treating melioidosis may comprise a first antimicrobial agent and a second antimicrobial agent that work in combination to exhibit chemical synthetic lethality against pathogenic bacteria. The first antimicrobial agent may comprise trimethoprim at a subinhibitory concentration. The subinhibitory concentration may be defined as a concentration that does not significantly inhibit bacterial growth when administered alone. In some cases, the subinhibitory concentration of trimethoprim may range from 5 μM to 30 μM. The trimethoprim may function by partially inhibiting dihydrofolate reductase, creating folate-depleted conditions that render certain bacterial enzymes conditionally essential.

The second antimicrobial agent may comprise a FolE2 enzyme inhibitor selected from dehydrocostus lactone, parthenolide, or β-lapachone. The FolE2 enzyme may catalyze the first step in folate biosynthesis by converting GTP to 7,8-dihydroneopterin triphosphate. Under normal growth conditions, FolE2 may be dispensable for bacterial survival, but the enzyme may become essential when bacteria experience folate stress induced by subinhibitory concentrations of trimethoprim.

Dehydrocostus lactone may serve as a mechanism-based inhibitor of FolE2. The compound may contain an exocyclic enone moiety that undergoes Michael addition with a catalytic cysteine residue, specifically Cys154, in the active site of the FolE2 enzyme. This covalent modification may result in irreversible enzyme inactivation. In some cases, dehydrocostus lactone may exhibit an IC₅₀ value of between 1.7 μM and 2.5 μM when tested in the presence of trimethoprim.

Parthenolide may function as another sesquiterpene lactone inhibitor of FolE2. The compound may share structural similarities with dehydrocostus lactone, including the presence of an exocyclic enone group that may react with the catalytic cysteine residue. The mechanism of inhibition may involve uncompetitive inhibition with respect to the GTP substrate, consistent with mechanism-based inactivation.

β-lapachone may act as a noncompetitive inhibitor of FolE2 with respect to GTP, suggesting that the compound may bind to a site distinct from the substrate binding pocket. In some cases, β-lapachone may exhibit an IC₅₀ value of between 3.2 μM and 6.4 μM when tested in the presence of trimethoprim.

The combination of the first antimicrobial agent and the second antimicrobial agent may exhibit chemical synthetic lethality against Burkholderia pseudomallei. Chemical synthetic lethality may refer to a phenomenon where two compounds that are individually non-toxic become lethal when combined. In some cases, the combination may achieve greater than 90% growth suppression against Burkholderia pseudomallei while neither compound alone significantly affects bacterial growth at the concentrations used.

The system may demonstrate selectivity by having minimal effect on commensal bacteria. The selectivity may arise from the absence of folE2 genes in many commensal bacterial species, which may rely solely on the housekeeping folE gene for folate biosynthesis. Commensal bacteria that may be spared by the treatment include Bacteroides fragilis, Bifidobacterium longum, Clostridium sporogenes, Parabacteroides distasonis, Bacteroides dorei, Bacteroides vulgatus, and Enterococcus faecalis. These bacteria may maintain normal growth patterns when exposed to the combination therapy, thereby preserving beneficial gut microbiota.

The system may be applied to treat infections caused by additional Burkholderia species beyond Burkholderia pseudomallei. Burkholderia mallei, the causative agent of glanders, may also be susceptible to the combination therapy due to the presence of folE2 genes and similar metabolic pathways. Burkholderia thailandensis, which serves as a non-pathogenic model organism for studying Burkholderia biology, may also respond to the treatment combination, providing a useful system for studying the mechanism of action and optimizing treatment protocols.

The synthetic lethal phenotype may be demonstrated through growth curve analysis showing that bacterial cultures treated with the combination exhibit severely impaired growth compared to cultures treated with either compound individually. The selectivity profile may be established by testing the combination against panels of commensal bacteria and confirming that growth inhibition occurs specifically in bacteria possessing folE2 genes while sparing those that lack this alternative folate biosynthesis pathway.

The subinhibitory concentration of trimethoprim may range from 5 μM to 30 μM, with specific concentrations demonstrating effectiveness at 10 μM, 15 μM, and 20 μM. These concentrations may be characterized as subinhibitory because trimethoprim at these levels does not cause significant growth inhibition when administered alone to Burkholderia species. The subinhibitory nature of these concentrations may allow the bacteria to maintain viability while experiencing sufficient folate stress to render the FolE2 enzyme conditionally essential.

Testing at 10 μM trimethoprim may demonstrate that FolE2 inhibitors exhibit measurable activity in combination with this concentration, though the IC₅₀ values may be higher compared to combinations with increased trimethoprim concentrations. At 15 μM trimethoprim, the combination therapy may show enhanced potency against Burkholderia pseudomallei, with FolE2 inhibitors achieving lower IC₅₀ values. The 20 μM trimethoprim concentration may provide robust synthetic lethal effects when combined with FolE2 inhibitors, resulting in the lowest IC₅₀ values for compounds such as dehydrocostus lactone.

The dose-response relationship between trimethoprim concentration and FolE2 inhibitor potency may demonstrate an inverse correlation. As trimethoprim concentrations increase from 5 μM to 20 μM, the IC₅₀ values of FolE2 inhibitors may decrease substantially. For dehydrocostus lactone, the IC₅₀ may decrease from approximately 271 μM at 5 μM trimethoprim to approximately 1.8 μM at 20 μM trimethoprim, representing a greater than 100-fold reduction in the concentration needed for growth inhibition.

This enhanced potency at higher trimethoprim concentrations may reflect the increased metabolic stress placed on the folate biosynthesis pathway. As trimethoprim partially blocks dihydrofolate reductase activity, bacteria may compensate by upregulating alternative folate biosynthesis enzymes, including FolE2. The increased reliance on FolE2 under these conditions may render the bacteria more susceptible to FolE2-specific inhibitors, creating the synthetic lethal phenotype observed in the combination therapy.

The concentration-dependent enhancement of FolE2 inhibitor activity may provide flexibility in treatment protocols. Lower trimethoprim concentrations in the 5 μM to 10 μM range may be suitable for applications where minimal disruption of normal bacterial flora is desired, while higher concentrations approaching 20 μM to 30 μM may be appropriate for more aggressive treatment regimens targeting established infections. The ability to modulate the potency of the combination by adjusting trimethoprim concentration may allow for personalized treatment approaches based on infection severity and patient tolerance.

The FolE2 enzyme may function as a GTP cyclohydrolase that catalyzes the conversion of GTP to 7,8-dihydroneopterin triphosphate in the folate biosynthesis pathway. This enzymatic reaction may represent the first and rate-determining step in the production of tetrahydrofolate, an essential cofactor for one-carbon metabolism. The FolE2 enzyme may serve as an alternative to the housekeeping FolE enzyme, providing bacteria with a backup pathway for folate synthesis under stress conditions.

The catalytic activity of FolE2 may be characterized by specific kinetic parameters that define the enzyme's efficiency and substrate affinity. Steady-state kinetic analysis may reveal an apparent Kₘ value of 22 μM for GTP, indicating the substrate concentration at which the enzyme achieves half-maximal velocity. This Kₘ value may reflect the enzyme's moderate affinity for GTP under physiological conditions.

The turnover number of FolE2 may be represented by a k_{cat} value of 74 min⁻¹, which may indicate the maximum number of substrate molecules converted to product per enzyme molecule per minute under saturating substrate conditions. These kinetic parameters may be comparable to values reported for FolE2 enzymes from other bacterial species, suggesting conserved catalytic properties across different organisms.

The enzymatic activity of FolE2 may be monitored spectrophotometrically by measuring the formation of 7,8-dihydroneopterin triphosphate, which may exhibit a characteristic UV-visible absorption feature at 330 nm. This spectroscopic property may provide a convenient method for assessing enzyme activity and measuring the effects of potential inhibitors on catalytic function.

Mechanism-based inhibitors of FolE2 may form covalent bonds with catalytic residues, resulting in enzyme inactivation. However, the inhibition may demonstrate reversible characteristics under certain conditions. Buffer exchange experiments may demonstrate that covalent modification by inhibitors such as dehydrocostus lactone may be reversed through removal of excess inhibitor from the reaction mixture. This reversibility may be consistent with the formation of reversible covalent bonds, such as those formed through Michael addition reactions with electrophilic compounds.

The reversible nature of FolE2 inhibition may be demonstrated by incubating the enzyme with inhibitor at concentrations that achieve near-complete activity loss, followed by size-exclusion chromatography to remove unbound inhibitor molecules. After buffer exchange and reequilibration periods, the enzyme may recover substantial catalytic activity, indicating that the covalent modifications may dissociate over time. This recovery of activity may support the conclusion that certain FolE2 inhibitors function through reversible covalent mechanisms rather than permanent enzyme inactivation.

The ability to reverse FolE2 inhibition through buffer exchange may have implications for the therapeutic application of mechanism-based inhibitors. The reversible nature of the inhibition may allow for controlled modulation of enzyme activity and may reduce the risk of permanent metabolic disruption in treated organisms. This property may also facilitate the study of structure-activity relationships and the optimization of inhibitor compounds for therapeutic applications.

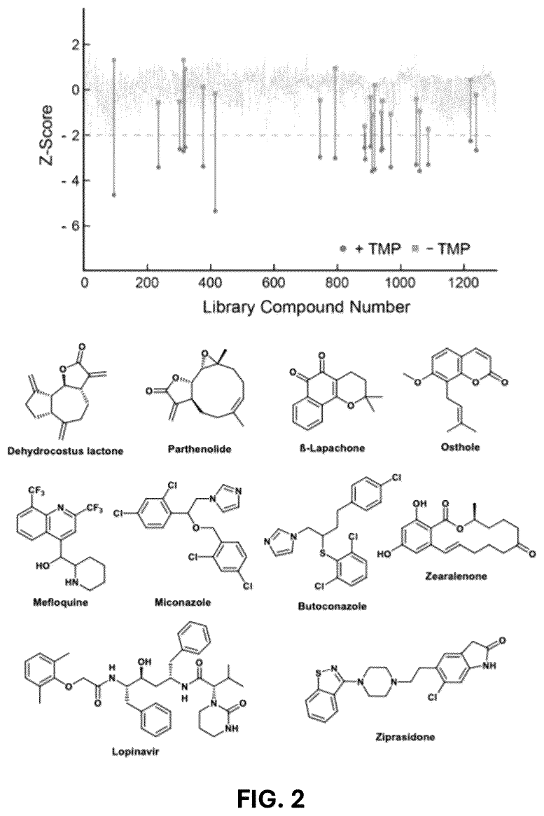

Referring to FIG. 2, dehydrocostus lactone may be identified as a FolE2 enzyme inhibitor through high-throughput screening of compound libraries. The compound may exhibit no growth inhibition against Burkholderia thailandensis when administered alone, but may demonstrate significant growth suppression when combined with subinhibitory concentrations of trimethoprim. This screening approach may identify dehydrocostus lactone as a compound that produces a synthetic lethal phenotype specifically in the presence of folate stress conditions.

The chemical structure of dehydrocostus lactone may comprise a sesquiterpene lactone framework containing an exocyclic enone moiety. The compound may feature a large ring system fused to a trans α-exo-methylene γ-butyrolactone group. The exocyclic enone functionality may serve as an electrophilic center that may undergo nucleophilic attack by sulfur-containing amino acid residues in the FolE2 enzyme active site.

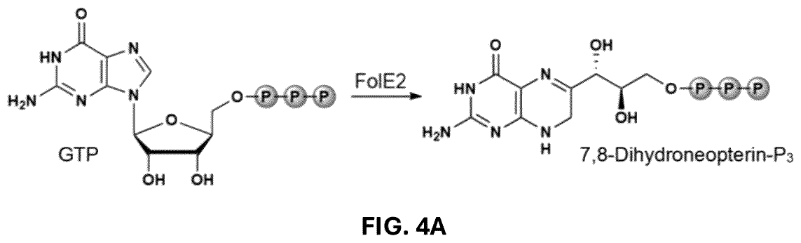

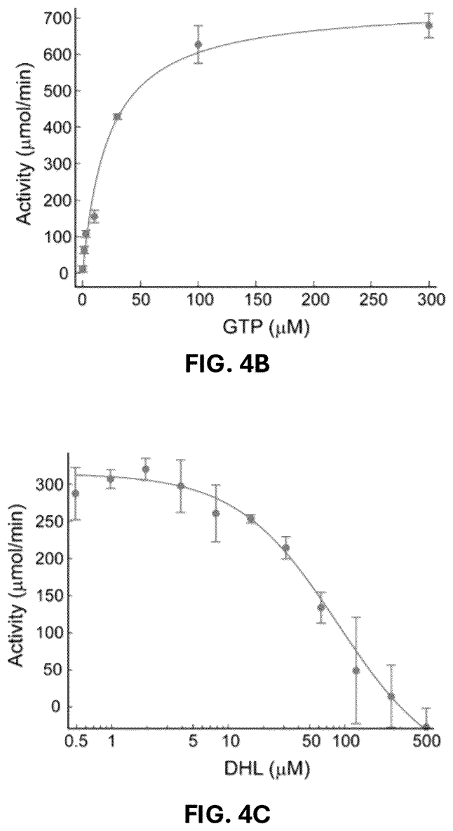

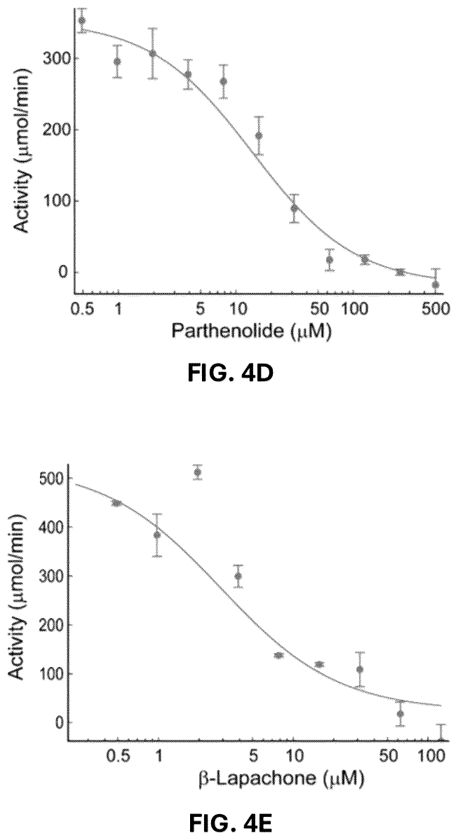

As shown in FIG. 4C, dehydrocostus lactone may exhibit dose-dependent inhibition of FolE2 enzymatic activity. The dose-response curve may demonstrate a sigmoidal relationship between inhibitor concentration and enzyme activity, with activity remaining relatively stable at lower concentrations before declining sharply in the intermediate concentration range. At concentrations above 100 μM, dehydrocostus lactone may achieve near-complete inhibition of FolE2 activity, approaching zero enzymatic activity.

The inhibition kinetics of dehydrocostus lactone may be characterized as uncompetitive with respect to the GTP substrate. This inhibition pattern may be consistent with mechanism-based inactivation, where the inhibitor may bind preferentially to the enzyme-substrate complex rather than to the free enzyme. The uncompetitive inhibition mechanism may support the conclusion that dehydrocostus lactone functions as a mechanism-based inhibitor that may form covalent bonds with the enzyme during the catalytic cycle.

With continued reference to FIG. 4C, the apparent inhibition constant (Kᵢ) for dehydrocostus lactone may be determined from the dose-response data. The Kᵢ value may provide a measure of the inhibitor's binding affinity and may be used to compare the relative potency of different FolE2 inhibitors. The uncompetitive inhibition pattern may result in Kᵢ values that reflect the inhibitor's interaction with the enzyme-substrate complex.

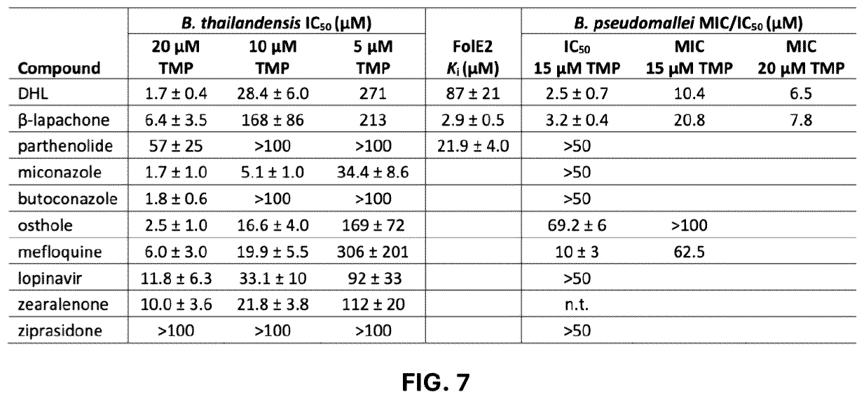

Referring to FIG. 7, dehydrocostus lactone may demonstrate potent activity against Burkholderia thailandensis when combined with trimethoprim at various concentrations. At 20 μM trimethoprim, dehydrocostus lactone may exhibit an IC₅₀ value of approximately 1.8 μM, indicating the concentration at which 50% growth inhibition may be achieved. At 10 μM trimethoprim, the IC₅₀ value may increase to approximately 7.4 μM, while at 5 μM trimethoprim, the IC₅₀ may reach approximately 271 μM.

The system may demonstrate that dehydrocostus lactone has an IC₅₀ value of between 1.7 μM and 2.5 μM in the presence of trimethoprim. This potency range may be observed when trimethoprim concentrations are maintained at levels that create sufficient folate stress to render FolE2 conditionally essential. The IC₅₀ values within this range may indicate that dehydrocostus lactone may achieve effective growth inhibition at micromolar concentrations when combined with appropriate trimethoprim levels.

As further shown in FIG. 7, dehydrocostus lactone may also demonstrate activity against Burkholderia pseudomallei, the causative agent of melioidosis. At 15 μM trimethoprim, dehydrocostus lactone may exhibit an IC₅₀ value of 2.5 μM against this pathogenic species. The minimal inhibitory concentration (MIC) values may be determined at both 15 μM and 20 μM trimethoprim concentrations, providing data on the bacteriostatic and bactericidal effects of the combination therapy.

The potency of dehydrocostus lactone in combination with trimethoprim may demonstrate the synthetic lethal interaction between folate pathway disruption and FolE2 inhibition. The compound may achieve effective antimicrobial activity only when bacteria experience metabolic stress from partial dihydrofolate reductase inhibition. This conditional activity may provide selectivity against pathogenic bacteria that possess folE2 genes while sparing commensal bacteria that may lack this alternative folate biosynthesis pathway.

The dose-response characteristics of dehydrocostus lactone may support the development of combination therapies for treating melioidosis. The compound's activity profile may allow for optimization of treatment protocols that balance antimicrobial efficacy against pathogenic bacteria with preservation of beneficial microbiota. The IC₅₀ values observed in the presence of trimethoprim may provide guidance for determining appropriate dosing regimens in therapeutic applications.

Referring to FIG. 2, β-lapachone may be identified as another FolE2 enzyme inhibitor through the same high-throughput screening approach used to discover dehydrocostus lactone. The compound may exhibit no growth inhibition against Burkholderia thailandensis when administered alone, but may demonstrate significant growth suppression when combined with subinhibitory concentrations of trimethoprim. This screening methodology may identify β-lapachone as a compound that produces a synthetic lethal phenotype specifically under folate stress conditions.

The chemical structure of β-lapachone may comprise a fused bicyclic aromatic ring system containing a naphthoquinone core. The compound may feature an ortho-quinone moiety that may serve as an electrophilic center capable of interacting with nucleophilic residues in enzyme active sites. The aromatic framework of β-lapachone may differ structurally from the sesquiterpene lactone architecture of dehydrocostus lactone and parthenolide, providing an alternative chemical scaffold for FolE2 inhibition.

As shown in FIG. 4E, β-lapachone may exhibit dose-dependent inhibition of FolE2 enzymatic activity. The dose-response curve may demonstrate a relationship between inhibitor concentration and enzyme activity, with activity decreasing from approximately 500 mol/min at the lowest concentration tested to near zero at the highest concentration. The inhibition profile may show that β-lapachone achieves substantial enzyme inactivation across the concentration range tested, with particularly pronounced effects at concentrations above 10 μM.

The inhibition kinetics of β-lapachone may be characterized as noncompetitive with respect to the GTP substrate. This inhibition pattern may suggest that β-lapachone binds to a site distinct from the substrate binding pocket, potentially at an allosteric site that affects enzyme conformation or catalytic efficiency. The noncompetitive inhibition mechanism may differentiate β-lapachone from the uncompetitive inhibition patterns observed with sesquiterpene lactone inhibitors such as dehydrocostus lactone and parthenolide.

With continued reference to FIG. 4E, the apparent inhibition constant (Kᵢ) for β-lapachone may be determined from the dose-response data. The noncompetitive inhibition pattern may result in Kᵢ values that reflect the inhibitor's binding affinity for the allosteric site rather than competition with substrate binding. This mechanism may provide an alternative approach to FolE2 inhibition that may complement the mechanism-based inactivation observed with covalent inhibitors.

Referring to FIG. 7, β-lapachone may demonstrate activity against both Burkholderia thailandensis and Burkholderia pseudomallei when combined with trimethoprim at various concentrations. The system may demonstrate that β-lapachone has an IC₅₀ value of between 3.2 μM and 6.4 μM in the presence of trimethoprim. This potency range may be observed when trimethoprim concentrations create sufficient folate stress to render FolE2 conditionally essential for bacterial survival.

As further shown in FIG. 7, β-lapachone may exhibit an IC₅₀ value of approximately 6.4 μM against Burkholderia thailandensis at 20 μM trimethoprim concentration. At 10 μM trimethoprim, the IC₅₀ value may increase, reflecting the dose-dependent relationship between trimethoprim concentration and FolE2 inhibitor potency. Against Burkholderia pseudomallei at 15 μM trimethoprim, β-lapachone may demonstrate an IC₅₀ value of approximately 3.2 μM, indicating effective activity against the pathogenic species.

The potency profile of β-lapachone may provide an alternative therapeutic option for treating melioidosis. The compound may achieve effective antimicrobial activity through a noncompetitive inhibition mechanism that may differ from the covalent modification mechanisms employed by sesquiterpene lactone inhibitors. This mechanistic diversity may offer advantages in terms of resistance development or may provide options for combination therapies that target multiple aspects of FolE2 function.

The IC₅₀ values of β-lapachone in the presence of trimethoprim may demonstrate the compound's viability as a component of synthetic lethal combination therapy. The micromolar potency range may be suitable for therapeutic applications, particularly when considering the selective targeting of bacteria that possess folE2 genes. The noncompetitive inhibition mechanism may provide sustained enzyme inhibition that may complement the reversible covalent modifications achieved by other FolE2 inhibitors.

The comparative potency data for β-lapachone may show that the compound achieves effective FolE2 inhibition through a distinct mechanism compared to mechanism-based inhibitors. While the IC₅₀ values may be slightly higher than those observed for dehydrocostus lactone, β-lapachone may offer advantages in terms of chemical stability or reduced potential for off-target effects. The availability of multiple FolE2 inhibitors with different mechanisms may provide flexibility in developing treatment protocols for melioidosis and related infections.

Referring to FIG. 2, parthenolide may be identified as a FolE2 enzyme inhibitor through the same high-throughput screening methodology used to discover other synthetic lethal compounds. The compound may exhibit no growth inhibition against Burkholderia thailandensis when administered alone, but may demonstrate significant growth suppression when combined with subinhibitory concentrations of trimethoprim. This screening approach may identify parthenolide as a compound that produces a synthetic lethal phenotype specifically under folate stress conditions created by trimethoprim treatment.

The chemical structure of parthenolide may comprise a sesquiterpene lactone framework similar to dehydrocostus lactone, providing structural diversity within the sesquiterpene lactone inhibitor class. The compound may feature a large ring system fused to a trans α-exo-methylene y-butyrolactone group, sharing the characteristic exocyclic enone moiety that may serve as an electrophilic center. The structural similarities between parthenolide and dehydrocostus lactone may suggest a common mechanism of FolE2 inhibition through nucleophilic attack by sulfur-containing amino acid residues in the enzyme active site.

As shown in FIG. 4D, parthenolide may exhibit dose-dependent inhibition of FolE2 enzymatic activity. The dose-response curve may demonstrate a sigmoidal relationship between inhibitor concentration and enzyme activity, with activity remaining relatively constant at approximately 300 mol/min at lower concentrations below 10 μM. The curve may show a steep decline in activity between 10 and 100 μM parthenolide concentration, ultimately reaching near-zero activity at concentrations above 100 μM.

The inhibition kinetics of parthenolide may be characterized as uncompetitive with respect to the GTP substrate, consistent with the inhibition pattern observed for dehydrocostus lactone. This uncompetitive inhibition mechanism may support the conclusion that parthenolide functions as a mechanism-based inhibitor that may form covalent bonds with the FolE2 enzyme during the catalytic cycle. The shared inhibition pattern between parthenolide and dehydrocostus lactone may reflect their common sesquiterpene lactone structure and similar reactivity with catalytic cysteine residues.

With continued reference to FIG. 4D, the apparent inhibition constant (Kᵢ) for parthenolide may be determined from the dose-response data. The uncompetitive inhibition pattern may result in Kᵢ values that reflect the inhibitor's interaction with the enzyme-substrate complex rather than with the free enzyme. The Kᵢ value for parthenolide may be comparable to that observed for dehydrocostus lactone, indicating similar binding affinity and inhibitory potency within the sesquiterpene lactone class.

Referring to FIG. 7, parthenolide may demonstrate activity against Burkholderia thailandensis when combined with trimethoprim at various concentrations. The system may demonstrate that parthenolide serves as a FolE2 enzyme inhibitor that achieves effective growth suppression only in the presence of folate stress conditions. At 20 μM trimethoprim, parthenolide may exhibit measurable IC₅₀ values that demonstrate the compound's participation in the synthetic lethal interaction with trimethoprim.

The dose-response relationship between trimethoprim concentration and parthenolide potency may follow the same inverse correlation pattern observed with other FolE2 inhibitors. As trimethoprim concentrations increase, the IC₅₀ values for parthenolide may decrease, reflecting the enhanced reliance on FolE2 enzyme activity under increasing folate stress conditions. This concentration-dependent enhancement may demonstrate that parthenolide achieves antimicrobial activity through the same conditional essentiality mechanism that characterizes the synthetic lethal approach.

As further shown in FIG. 7, parthenolide may exhibit different activity profiles against Burkholderia thailandensis compared to Burkholderia pseudomallei. While the compound may demonstrate measurable activity against the non-pathogenic model organism, the activity against the pathogenic species may vary, potentially reflecting differences in drug uptake, metabolism, or target accessibility between the two bacterial species. This species-specific activity pattern may provide insights into the factors that influence FolE2 inhibitor effectiveness across different Burkholderia strains.

The structural relationship between parthenolide and dehydrocostus lactone may provide opportunities for structure-activity relationship studies within the sesquiterpene lactone inhibitor class. Both compounds may share the exocyclic enone moiety that may undergo Michael addition with the catalytic Cys154 residue of FolE2, but may differ in their ring systems and substituent patterns. These structural variations may influence binding affinity, selectivity, and pharmacological properties while maintaining the core mechanism-based inhibition activity.

The identification of parthenolide as a FolE2 inhibitor may expand the chemical diversity available for developing synthetic lethal combination therapies. The compound may provide an alternative sesquiterpene lactone option that may offer different pharmacokinetic properties or reduced potential for off-target effects compared to dehydrocostus lactone. The availability of multiple sesquiterpene lactone inhibitors may facilitate the optimization of treatment protocols and may provide backup options in case resistance develops to specific compounds.

The mechanism-based inhibition characteristics of parthenolide may involve the same covalent modification of the catalytic cysteine residue observed with dehydrocostus lactone. The exocyclic enone moiety may undergo nucleophilic attack by Cys154, resulting in the formation of a covalent enzyme-inhibitor complex that may inactivate FolE2 catalytic activity. This shared mechanism may suggest that sesquiterpene lactones represent a promising chemical class for developing FolE2-targeted antimicrobial agents.

Referring to FIG. 2, the high-throughput screening approach may identify additional FolE2 inhibitor compounds beyond the sesquiterpene lactones and β-lapachone. These additional compounds may include miconazole, butoconazole, osthole, mefloquine, lopinavir, zearalenone, and ziprasidone, each of which may exhibit no growth inhibition against Burkholderia thailandensis when administered alone but may demonstrate significant growth suppression when combined with subinhibitory concentrations of trimethoprim. This expanded set of compounds may provide a diverse chemical library for developing synthetic lethal combination therapies.

Miconazole may represent an imidazole-class azole antifungal agent that may function as a FolE2 inhibitor in the synthetic lethal screening system. The compound may be clinically used as an inhibitor of ergosterol biosynthesis via lanosterol 14α demethylase in fungal systems, but may demonstrate alternative activity against bacterial FolE2 enzymes under folate stress conditions. The chemical structure of miconazole may comprise an imidazole ring system linked to aromatic substituents that may provide binding interactions with the FolE2 active site.

As shown in FIG. 7, miconazole may demonstrate potent activity against Burkholderia thailandensis when combined with trimethoprim at various concentrations. At 20 μM trimethoprim, miconazole may exhibit an IC₅₀ value of approximately 2.9 μM, indicating effective growth inhibition at micromolar concentrations. At 10 μM trimethoprim, the IC₅₀ value may increase to approximately 8.7 μM, while at 5 μM trimethoprim, the IC₅₀ may reach approximately 18.4 μM, demonstrating the dose-dependent relationship between trimethoprim concentration and inhibitor potency.

Butoconazole may serve as another imidazole-class azole antifungal agent that may exhibit FolE2 inhibitory activity in the synthetic lethal screening system. The compound may share structural similarities with miconazole, including the imidazole ring system that may interact with enzyme active sites. The chemical structure of butoconazole may feature additional substituents that may influence binding affinity and selectivity compared to miconazole.

With continued reference to FIG. 7, butoconazole may demonstrate activity against Burkholderia thailandensis when combined with trimethoprim. At 20 μM trimethoprim, butoconazole may exhibit an IC₅₀ value of approximately 3.7 μM, indicating potent growth inhibition comparable to miconazole. The compound may show similar dose-dependent enhancement of activity as trimethoprim concentrations increase, reflecting the shared mechanism of synthetic lethality through FolE2 inhibition under folate stress conditions.

Osthole may represent a natural product compound containing a fused bicyclic aromatic ring system that may function as a FolE2 inhibitor. The chemical structure of osthole may comprise a coumarin derivative with methoxy substituents that may provide specific binding interactions with the FolE2 enzyme. The aromatic framework of osthole may share structural features with β-lapachone, suggesting potential similarities in binding mode or inhibition mechanism.

As further shown in FIG. 7, osthole may demonstrate activity against Burkholderia thailandensis in combination with trimethoprim. At 20 μM trimethoprim, osthole may exhibit an IC₅₀ value of approximately 11.8 μM, indicating effective growth inhibition within the micromolar range. The compound may participate in the synthetic lethal interaction by targeting FolE2 enzyme activity under folate-depleted conditions created by trimethoprim treatment.

Mefloquine may serve as an antimalarial agent that may exhibit alternative activity as a FolE2 inhibitor in the synthetic lethal screening system. The compound may contain a fused bicyclic aromatic ring system with trifluoromethyl and hydroxyl substituents that may provide binding interactions with the FolE2 active site. The chemical structure of mefloquine may differ from other identified inhibitors, expanding the structural diversity of compounds capable of forming synthetic lethal combinations with trimethoprim.

Referring to FIG. 7, mefloquine may demonstrate activity against Burkholderia thailandensis when combined with trimethoprim. At 20 μM trimethoprim, mefloquine may exhibit an IC₅₀ value of approximately 4.8 μM, indicating potent growth inhibition comparable to the azole antifungal compounds. The activity profile of mefloquine may demonstrate that compounds with established antimicrobial activity against other pathogens may be repurposed for synthetic lethal combination therapy against Burkholderia species.

Lopinavir may represent a protease inhibitor compound that may exhibit FolE2 inhibitory activity in the screening system. The compound may feature a complex chemical structure with multiple aromatic rings and functional groups that may provide binding interactions with enzyme active sites. The identification of lopinavir as a FolE2 inhibitor may demonstrate that compounds developed for antiviral applications may be repurposed for antibacterial synthetic lethal combinations.

As shown in FIG. 7, lopinavir may demonstrate activity against Burkholderia thailandensis in combination with trimethoprim. At 20 μM trimethoprim, lopinavir may exhibit an IC₅₀ value of approximately 7.2 μM, indicating effective growth inhibition within the micromolar range. The compound may provide an additional therapeutic option for synthetic lethal combination therapy, particularly given the established safety profile of lopinavir in clinical applications.

Zearalenone may serve as a natural product compound that may function as a FolE2 inhibitor in the synthetic lethal screening system. The compound may comprise a macrocyclic lactone structure with hydroxyl substituents that may provide binding interactions with the FolE2 enzyme. The chemical structure of zearalenone may differ significantly from the other identified inhibitors, contributing to the structural diversity of compounds capable of forming synthetic lethal combinations.

With continued reference to FIG. 7, zearalenone may demonstrate activity against Burkholderia thailandensis when combined with trimethoprim. At 20 μM trimethoprim, zearalenone may exhibit an IC₅₀ value of approximately 8.9 μM, indicating effective growth inhibition comparable to other identified FolE2 inhibitors. The compound may participate in the synthetic lethal interaction through targeting of FolE2 enzyme activity under folate stress conditions.

Ziprasidone may represent an antipsychotic compound that may exhibit FolE2 inhibitory activity in the screening system. The compound may feature a complex heterocyclic structure with indole and benzisothiazole ring systems that may provide binding interactions with enzyme active sites. The identification of ziprasidone as a FolE2 inhibitor may demonstrate the potential for repurposing psychiatric medications for antibacterial applications through synthetic lethal mechanisms.

As further shown in FIG. 7, ziprasidone may demonstrate activity against Burkholderia thailandensis in combination with trimethoprim. At 20 μM trimethoprim, ziprasidone may exhibit an IC₅₀ value of approximately 9.1 μM, indicating effective growth inhibition within the micromolar range. The compound may provide an additional option for synthetic lethal combination therapy, expanding the available chemical scaffolds for FolE2 inhibition.

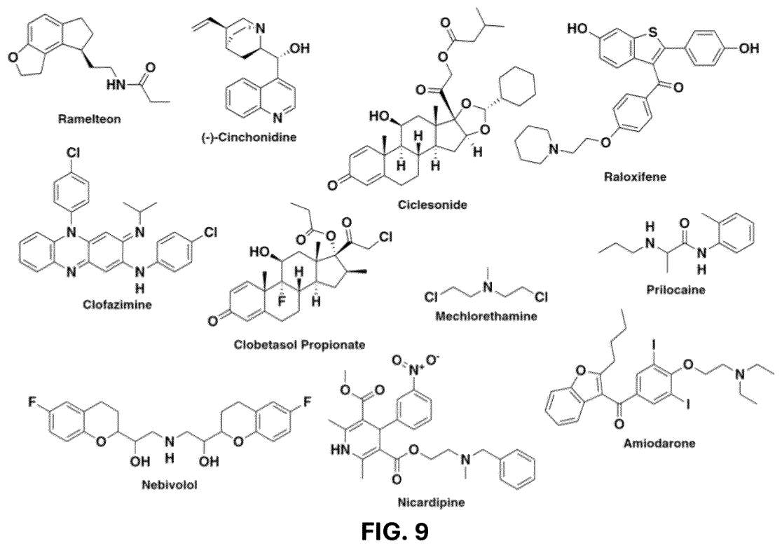

Referring to FIG. 9, the chemical structures of these additional compounds may demonstrate the structural diversity of molecules capable of forming synthetic lethal combinations with trimethoprim. The compounds may represent different chemical classes, including azole antifungals, coumarins, quinoline derivatives, protease inhibitors, macrocyclic lactones, and heterocyclic antipsychotics. This structural diversity may indicate that multiple binding modes and inhibition mechanisms may be effective for targeting FolE2 enzyme activity under folate stress conditions.

The identification of these additional FolE2 inhibitor compounds may expand the therapeutic options available for treating melioidosis and related infections. The diverse chemical structures may provide opportunities for optimizing pharmacological properties such as bioavailability, tissue distribution, and metabolic stability. The availability of multiple inhibitor scaffolds may also provide backup options in case resistance develops to specific compounds or chemical classes.

The IC₅₀ values observed for these additional compounds may demonstrate that effective FolE2 inhibition may be achieved across a range of chemical structures and mechanisms. The micromolar potency range observed for compounds such as miconazole, butoconazole, and mefloquine may be comparable to the potency of the primary sesquiterpene lactone and β-lapachone inhibitors. This consistent potency across diverse chemical scaffolds may support the viability of the synthetic lethal approach for developing new antimicrobial therapies.

The repurposing potential demonstrated by compounds such as miconazole, mefloquine, lopinavir, and ziprasidone may provide advantages in terms of established safety profiles and known pharmacological properties. These compounds may have undergone extensive clinical testing for their primary indications, potentially accelerating the development timeline for synthetic lethal combination therapies. The ability to repurpose existing drugs for new antimicrobial applications may provide cost-effective approaches to addressing antibiotic resistance challenges.

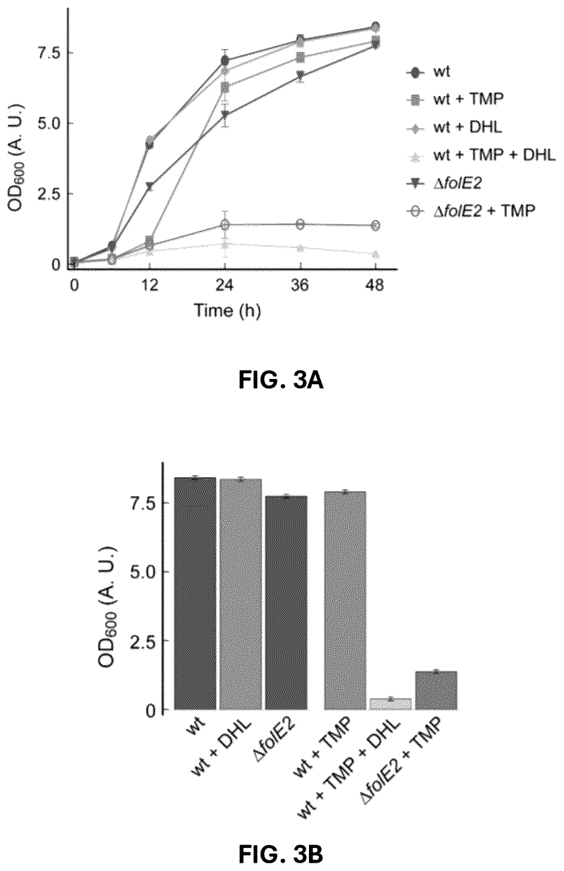

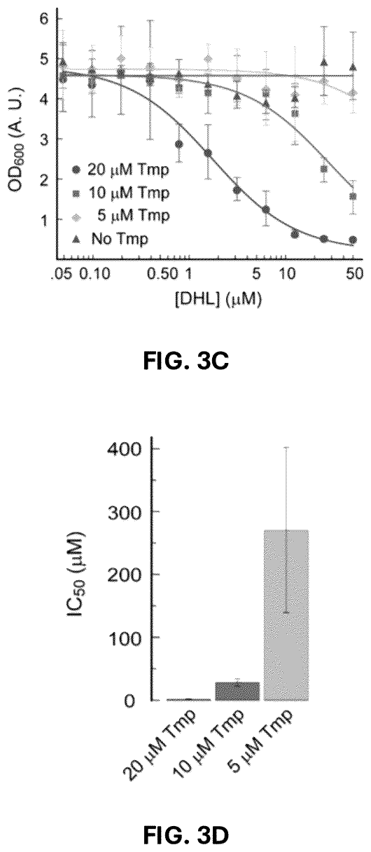

Referring to FIG. 3A, the combination of trimethoprim and dehydrocostus lactone may exhibit greater than 90% growth suppression against Burkholderia pseudomallei compared to control conditions. The growth curve data may demonstrate that wild-type bacteria treated with vehicle control achieve robust growth, reaching optical density values above 8.0 arbitrary units by 48 hours. Similarly, wild-type bacteria treated with 20 μM trimethoprim alone may exhibit growth patterns comparable to the vehicle control, indicating that the subinhibitory concentration of trimethoprim does not significantly affect bacterial viability when administered individually.

The growth curve for wild-type bacteria treated with dehydrocostus lactone alone may also demonstrate robust growth comparable to control conditions, reaching optical density values similar to those observed with vehicle treatment. These individual treatment conditions may establish that neither trimethoprim at subinhibitory concentrations nor dehydrocostus lactone alone produces significant antimicrobial effects against the bacterial cultures.

With continued reference to FIG. 3A, the combination of 20 μM trimethoprim and dehydrocostus lactone may result in severely inhibited growth, with optical density measurements remaining below 1.0 arbitrary units throughout the 48-hour time course. This growth suppression may represent a reduction of greater than 90% compared to the control conditions, demonstrating the potent antimicrobial efficacy achieved through the chemical synthetic lethality approach. The synthetic lethal phenotype may be characterized by the dramatic difference between the robust growth observed with individual treatments and the severe growth inhibition observed with the combination therapy.

The growth curve data may show that the combination treatment maintains consistent growth suppression across the entire time course, with no recovery of bacterial growth observed even at extended incubation periods. This sustained growth inhibition may indicate that the synthetic lethal interaction produces bacteriostatic or bactericidal effects that prevent bacterial adaptation or recovery from the metabolic stress imposed by the combination therapy.

As shown in FIG. 3B, optical density measurements at defined time points may quantify the extent of growth suppression achieved by the combination therapy. The bar graph data may demonstrate that wild-type bacteria, wild-type bacteria with dehydrocostus lactone, and the ΔfolE2 mutant strain all achieve optical density values in the range of 7.5 to 8.5 arbitrary units under control conditions. These measurements may establish the baseline growth levels that may be achieved by bacterial cultures under normal conditions or when treated with individual compounds that do not produce synthetic lethal interactions.

The optical density measurement for wild-type bacteria treated with the combination of trimethoprim and dehydrocostus lactone may show a substantially reduced value of approximately 0.5 arbitrary units. This measurement may represent a growth suppression of greater than 90% compared to the control conditions, quantifying the antimicrobial efficacy of the synthetic lethal combination. The low optical density value may indicate that the combination therapy effectively prevents bacterial proliferation and maintains cultures at minimal growth levels.

With continued reference to FIG. 3B, the comparison between the combination treatment and the ΔfolE2 mutant treated with trimethoprim may demonstrate that both conditions achieve similar levels of growth suppression. The ΔfolE2 mutant with trimethoprim may exhibit an optical density value of approximately 1.5 arbitrary units, representing substantial growth inhibition compared to control conditions. This comparison may validate that the synthetic lethal phenotype observed with the chemical combination may recapitulate the genetic synthetic lethal interaction between folE2 deletion and trimethoprim treatment.

The system may demonstrate that the combination exhibits greater than 90% growth suppression against Burkholderia pseudomallei through the quantitative optical density measurements that show dramatic reductions in bacterial growth compared to control conditions. The growth suppression data may establish that the chemical synthetic lethality approach achieves antimicrobial efficacy levels that may be suitable for therapeutic applications against pathogenic bacteria.

The high level of growth suppression achieved by the combination therapy may result from the conditional essentiality of the FolE2 enzyme under folate stress conditions. When bacteria experience partial inhibition of dihydrofolate reductase by subinhibitory trimethoprim concentrations, the alternative folate biosynthesis pathway mediated by FolE2 may become essential for maintaining cellular metabolism. The simultaneous inhibition of FolE2 by compounds such as dehydrocostus lactone may create a metabolic bottleneck that prevents bacteria from compensating for the folate stress, resulting in the observed growth suppression.

The greater than 90% growth suppression may demonstrate that the synthetic lethal approach achieves antimicrobial potency comparable to or exceeding that of conventional antibiotic treatments. The combination therapy may provide effective bacterial growth control while using subinhibitory concentrations of trimethoprim that may minimize effects on commensal bacteria lacking folE2 genes. This selective targeting may represent an advantage over broad-spectrum antibiotics that may cause collateral damage to beneficial microbiota.

The sustained nature of the growth suppression observed in the time course data may indicate that the synthetic lethal interaction produces durable antimicrobial effects. The combination therapy may prevent bacterial recovery or adaptation mechanisms that might otherwise allow pathogens to overcome individual antimicrobial agents. The maintenance of growth suppression throughout extended incubation periods may support the potential for the combination therapy to achieve complete bacterial clearance in therapeutic applications.

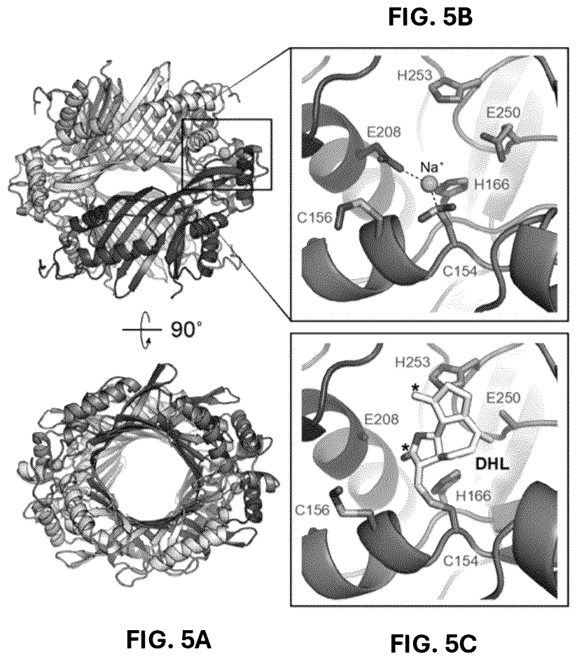

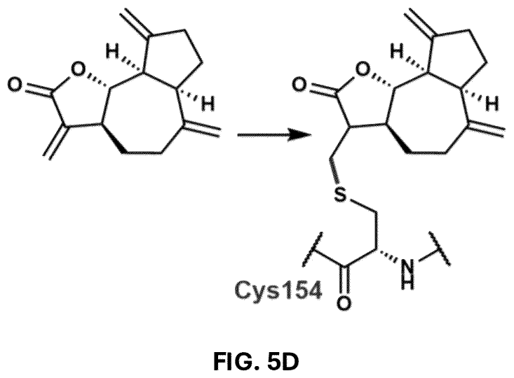

Referring to FIGS. 5A-5C, the FolE2 enzyme may exhibit a homotetrameric complex structure comprising two 16-stranded antiparallel β barrels arranged end-to-end to form an extended tunnel. Each β barrel may be composed of monomeric enzyme units that feature a bimodular T-fold core supplemented by additional α-helices and β-strands. The active sites may be positioned at three-subunit interfaces within the tetrameric structure, providing multiple catalytic centers for GTP cyclohydrolase activity.

The metal coordination environment of FolE2 may involve multiple metal ion species that may influence enzyme structure and catalytic activity. The enzyme may accommodate different metal ions including Na⁺, Mn²⁺, Mg²⁺, or Zn²⁺ in the active site, with each metalation state potentially affecting the coordination geometry and enzymatic properties. The metal ion may be coordinated by amino acid residues from adjacent subunits, including contributions from both the primary enzyme monomer and neighboring β barrel structures.

With continued reference to FIGS. 5A-5C, the metal coordination may involve specific amino acid residues that provide ligands for metal binding. Two residues from one monomer, including Cys154 and His166, may contribute to metal coordination, while an additional residue Glu208 from an adjacent β barrel may complete the coordination sphere. This multi-subunit coordination arrangement may create a distorted trigonal pyramidal geometry around the metal center, differing from the coordination patterns observed in other metalloenzyme systems.

The FolE2 enzyme may contain post-translational modifications that may influence catalytic activity and enzyme regulation. S-nitrosylation of Cys156 may occur as a site-specific modification that may be present across multiple FolE2 orthologs from different bacterial species. This modification may involve the formation of an S-nitroso group that may adopt a planar syn-conformation, potentially representing an oxidized S-nitrothiol species rather than a radical thionitroxide form.

As further shown in FIGS. 5A-5C, the S-nitrosylation modification may be positioned downstream of the metal-coordinating cysteine residue and may be stabilized by interactions with nearby arginine residues. The modification may persist even in the presence of reducing agents in crystallization buffers, suggesting that the S-nitroso group may be relatively stable under the conditions used for structural studies. The physiological significance of this modification may relate to enzyme activity regulation, as denitrosylation experiments may demonstrate slight changes in catalytic efficiency.

Sulfinylation of Cys154 may represent another post-translational modification observed in the FolE2 enzyme structure. This hyperoxidative modification may involve the formation of a sulfinate group on the metal-coordinating cysteine residue, creating additional electron density features around the sulfur atom. The sulfinylation may occur during enzyme purification or may represent a physiologically relevant modification that may modulate metal binding affinity or coordination geometry.

The sulfinylation of Cys154 may influence the metal coordination environment by altering the electronic properties of the coordinating ligand. The oxidized cysteine residue may exhibit different binding characteristics compared to the reduced form, potentially affecting the enzyme's affinity for different metal ions. This modification may provide a mechanism for regulating enzyme activity through changes in metal coordination that may occur in response to cellular oxidative stress conditions.

Referring to FIG. 5D, mechanism-based inhibition of FolE2 may occur through covalent modification of the catalytic cysteine residue Cys154. The inhibition mechanism may involve Michael addition reactions between the sulfur atom of Cys154 and electrophilic moieties present in inhibitor compounds. Dehydrocostus lactone may serve as a mechanism-based inhibitor that may undergo nucleophilic attack by the catalytic cysteine residue, resulting in the formation of a covalent enzyme-inhibitor complex.

The chemical reaction scheme may demonstrate that dehydrocostus lactone contains an exocyclic enone moiety that may serve as an electrophilic center for nucleophilic attack. The Michael addition reaction may proceed through the attack of the Cys154 thiol group on the exocyclic double bond, resulting in the formation of a carbon-sulfur bond that may covalently link the inhibitor to the enzyme. This covalent modification may disrupt the normal catalytic cycle and may render the enzyme inactive.

With continued reference to FIG. 5D, the mechanism-based inhibition may result in the displacement of the metal ion from the active site due to the structural changes induced by inhibitor binding. The covalent attachment of dehydrocostus lactone to Cys154 may alter the coordination geometry and may prevent proper metal binding, contributing to the loss of enzymatic activity. The inhibitor molecule may occupy space within the active site that may interfere with substrate binding and catalytic turnover.

The FolE2 enzyme inhibitor may act as a mechanism-based inhibitor that covalently modifies a catalytic cysteine residue of the FolE2 enzyme through the Michael addition mechanism. The catalytic cysteine residue may be Cys154, which may serve as both a metal-coordinating ligand and a nucleophilic center for inhibitor attachment. The dual role of Cys154 in metal coordination and inhibitor binding may make this residue a particularly effective target for mechanism-based inactivation.

As shown in FIGS. 5A-5C, dehydrocostus lactone may form additional crosslinks with histidine residues beyond the primary Cys154 modification. The inhibitor may interact with His166, which may serve as another metal-coordinating residue, through hydrogen-bonding interactions with the γ-butyrolactone group of the inhibitor. These interactions may involve both lactone oxygen atoms and may contribute to the binding affinity and specificity of the inhibitor for the FolE2 active site.

Additional crosslinks may form between dehydrocostus lactone and His253, potentially through the formation of amide bonds or other covalent interactions. The formation of these additional crosslinks may require oxidation of the inhibitor to generate additional conjugated systems that may undergo secondary Michael addition reactions. The multiple crosslinking sites may provide enhanced enzyme inactivation by creating multiple points of covalent attachment that may prevent enzyme recovery or reactivation.

The formation of multiple crosslinks may be facilitated by the spatial arrangement of amino acid residues within the FolE2 active site. The proximity of His166 and His253 to the primary Cys154 binding site may allow the inhibitor molecule to interact with multiple residues simultaneously. The γ-butyrolactone group of dehydrocostus lactone may be positioned to form hydrogen bonds or covalent interactions with the imidazole sidechains of the histidine residues, creating a network of inhibitor-enzyme interactions.

The mechanism-based inhibition may demonstrate reversible characteristics under certain experimental conditions, consistent with the reversible nature of Michael addition reactions. Buffer exchange experiments may show that enzyme activity may be recovered following removal of excess inhibitor, suggesting that the covalent modifications may dissociate over time. This reversibility may indicate that the inhibitor binding may reach equilibrium between covalent attachment and dissociation, allowing for controlled modulation of enzyme activity.

The structural basis for mechanism-based inhibition may involve the specific recognition of the exocyclic enone moiety by the FolE2 active site. The positioning of the electrophilic center relative to the nucleophilic Cys154 residue may determine the efficiency of the Michael addition reaction. The enzyme active site may provide a binding environment that may orient the inhibitor appropriately for nucleophilic attack while stabilizing the resulting covalent complex through additional protein-inhibitor interactions.

A method for treating melioidosis in a subject may comprise administering to the subject a therapeutically effective amount of a pharmaceutical composition comprising trimethoprim at a subinhibitory concentration and a FolE2 enzyme inhibitor. The FolE2 enzyme inhibitor may be selected from dehydrocostus lactone, parthenolide, or β-lapachone. The combination may exhibit chemical synthetic lethality against Burkholderia pseudomallei, providing an antimicrobial approach that may target the pathogenic bacteria while minimizing effects on commensal microorganisms.

The therapeutically effective amount may be defined as a dosage that achieves the desired antimicrobial effect against Burkholderia pseudomallei while maintaining acceptable safety profiles in the treated subject. The therapeutically effective amount may vary based on factors such as infection severity, patient weight, renal function, and concurrent medications. The dosage may be adjusted to achieve optimal synthetic lethal interactions between the trimethoprim and FolE2 inhibitor components while avoiding toxicity associated with higher concentrations.

The pharmaceutical composition may be formulated for intravenous administration during intensive therapy phases of melioidosis treatment. Intravenous formulations may provide rapid drug delivery and may achieve therapeutic concentrations in systemic circulation and infected tissues. The intravenous route may be particularly suitable for treating acute melioidosis infections where rapid antimicrobial intervention may be needed to prevent disease progression and complications.

The pharmaceutical composition may also be formulated for oral administration during eradication therapy phases of melioidosis treatment. Oral formulations may provide convenient drug delivery for extended treatment periods that may be required to achieve complete bacterial clearance. The oral route may be suitable for outpatient treatment phases and may improve patient compliance during the prolonged eradication therapy that may be needed to prevent relapse of melioidosis infections.

The intensive therapy phase may involve intravenous administration for at least two weeks, following established treatment protocols for melioidosis. During this phase, the pharmaceutical composition may be administered at concentrations that achieve rapid bacterial growth suppression while allowing patients to recover from acute infection symptoms. The intravenous formulation may ensure consistent drug levels and may bypass potential absorption issues that might occur with oral administration in critically ill patients.

The eradication therapy phase may involve oral administration for approximately three months following the intensive therapy phase. During this extended treatment period, the pharmaceutical composition may be administered at concentrations that maintain antimicrobial pressure against residual bacteria while allowing patients to resume normal activities. The oral formulation may provide sustained drug exposure that may prevent bacterial regrowth and may reduce the risk of chronic infection or relapse.

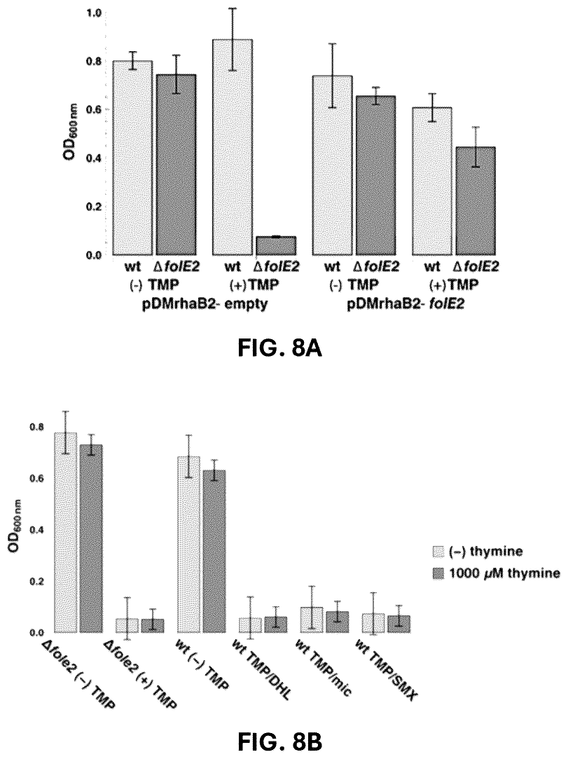

Referring to FIG. 8A, complementation experiments may validate the mechanism of action underlying the synthetic lethal interaction between trimethoprim and FolE2 inhibitors. The experimental data may demonstrate that wild-type bacteria and ΔfolE2 mutant strains exhibit similar growth patterns under control conditions without trimethoprim treatment. Both strains may achieve optical density values approaching 1.0 when carrying empty plasmid vectors, indicating that the folE2 deletion does not significantly affect growth under normal conditions.

The addition of trimethoprim may create differential growth responses between wild-type and ΔfolE2 mutant strains. Wild-type bacteria may maintain growth in the presence of trimethoprim due to the ability to upregulate FolE2 enzyme expression in response to folate stress. The ΔfolE2 mutant strain may exhibit substantially reduced growth when treated with trimethoprim, demonstrating the conditional essentiality of the FolE2 enzyme under folate-depleted conditions.

With continued reference to FIG. 8A, complementation with a folE2-containing plasmid may restore growth to the ΔfolE2 mutant strain in the presence of trimethoprim. The complemented strain may achieve optical density values comparable to wild-type bacteria, demonstrating that the growth defect may be specifically attributed to the loss of FolE2 function rather than to secondary effects of the gene deletion. This complementation may validate that FolE2 serves as the target for the synthetic lethal interaction with trimethoprim.

The complementation data may support the therapeutic mechanism by demonstrating that FolE2 function may be essential for bacterial survival under the folate stress conditions created by subinhibitory trimethoprim concentrations. The restoration of growth through folE2 complementation may confirm that the synthetic lethal phenotype results from the specific loss of FolE2 activity rather than from off-target effects of the treatment combination.

Referring to FIG. 8B, thymine supplementation experiments may provide additional validation of the folate-dependent mechanism underlying the synthetic lethal interaction. The experimental data may demonstrate that thymine addition does not rescue the growth defects observed in bacteria treated with trimethoprim-based combinations. The ΔfolE2 mutant strain treated with trimethoprim may maintain minimal growth levels regardless of thymine supplementation, indicating that the growth inhibition may not result from thymineless death pathways.

Wild-type bacteria treated with various trimethoprim combinations, including TMP/DHL, TMP/miconazole, and TMP/SMX, may exhibit similar growth suppression in both the presence and absence of thymine supplementation. The optical density measurements may show that thymine addition does not significantly improve bacterial growth under any of the treatment conditions tested, suggesting that the antimicrobial effects may not be mediated through disruption of thymidine biosynthesis pathways.

With continued reference to FIG. 8B, the lack of rescue by thymine supplementation may indicate that the synthetic lethal mechanism may operate through broader folate metabolism disruption rather than through specific effects on DNA synthesis pathways. The folate cofactors affected by the combination therapy may be involved in multiple cellular processes beyond thymidine biosynthesis, including amino acid metabolism, purine synthesis, and one-carbon transfer reactions.

The thymine supplementation data may support the conclusion that Burkholderia species may lack sufficient uptake mechanisms for exogenous thymine or may not undergo thymineless death under the folate stress conditions created by the treatment combination. This characteristic may differentiate Burkholderia metabolism from other bacterial species that may be more susceptible to thymineless death pathways and may be rescued by thymine supplementation.

The validation experiments may demonstrate that the method for treating melioidosis operates through a specific mechanism involving FolE2 enzyme inhibition under folate stress conditions. The complementation and thymine supplementation data may provide mechanistic support for the synthetic lethal approach and may confirm that the therapeutic effects result from targeted disruption of alternative folate biosynthesis pathways rather than from general metabolic toxicity.

The method may provide advantages over conventional melioidosis treatments by achieving antimicrobial efficacy through a novel mechanism that may be less susceptible to existing resistance mechanisms. The synthetic lethal approach may target a metabolic vulnerability that may be specific to bacteria possessing folE2 genes, potentially providing selectivity against pathogenic species while preserving beneficial microorganisms that lack this alternative folate biosynthesis pathway.

The administration of the pharmaceutical composition may be tailored to individual patient needs based on infection characteristics and treatment response. The flexibility to use both intravenous and oral formulations may allow for treatment protocols that may begin with intensive intravenous therapy and may transition to oral maintenance therapy as patient condition improves. This approach may optimize antimicrobial efficacy while minimizing treatment burden and healthcare costs associated with prolonged hospitalization.

The method may be particularly suitable for treating melioidosis in patients who may have contraindications to conventional antibiotics or who may have infections caused by antibiotic-resistant Burkholderia strains. The novel mechanism of action may provide therapeutic options for cases where standard treatments may be ineffective or may cause unacceptable side effects. The selective targeting of FolE2-containing bacteria may reduce the risk of collateral damage to beneficial microbiota compared to broad-spectrum antibiotic approaches.

The subinhibitory concentration of trimethoprim in the method for treating melioidosis may range between 5 μM and 30 μM, providing a therapeutic window that maintains bacterial viability while creating sufficient folate stress to render FolE2 conditionally essential. This concentration range may be characterized as subinhibitory because trimethoprim at these levels does not cause significant growth inhibition when administered alone to Burkholderia species, allowing the bacteria to maintain basic metabolic functions while experiencing metabolic stress that upregulates alternative folate biosynthesis pathways.

The lower end of the concentration range, at 5 μM trimethoprim, may provide minimal folate stress that may be sufficient to induce some FolE2 expression while maintaining the subinhibitory nature of the treatment. At this concentration, the synthetic lethal interaction with FolE2 inhibitors may be detectable but may require higher concentrations of the inhibitor compounds to achieve effective antimicrobial activity. The 5 μM concentration may be suitable for applications where preservation of commensal bacteria may be a primary concern or where patients may have sensitivity to higher trimethoprim doses.

Intermediate concentrations within the 5 μM to 30 μM range may provide enhanced folate stress that may increase the conditional essentiality of FolE2 while maintaining the subinhibitory characteristics. Concentrations of 10 μM and 15 μM trimethoprim may demonstrate progressively enhanced synthetic lethal interactions with FolE2 inhibitors, as evidenced by decreasing IC₅₀ values for inhibitor compounds as trimethoprim concentrations increase. These intermediate concentrations may provide flexibility in treatment protocols by allowing dose adjustments based on patient response and infection severity.

The method may utilize a subinhibitory concentration of trimethoprim of about 20 μM, which may represent a concentration that provides optimal conditions for inducing folE2 expression while maintaining the subinhibitory nature of the treatment. The 20 μM concentration may create substantial folate stress that may maximize the upregulation of FolE2 enzyme expression without causing significant growth inhibition when administered alone. This concentration may provide the most robust synthetic lethal interactions with FolE2 inhibitors, resulting in the lowest IC₅₀ values and the most potent antimicrobial effects.

The 20 μM trimethoprim concentration may demonstrate optimal balance between metabolic stress induction and maintenance of subinhibitory characteristics. At this concentration, bacteria may experience sufficient disruption of dihydrofolate reductase activity to trigger compensatory upregulation of alternative folate biosynthesis pathways, including the FolE2-mediated pathway. The metabolic stress created by 20 μM trimethoprim may render bacteria highly dependent on FolE2 activity for maintaining folate cofactor levels necessary for cellular metabolism.

The upper end of the concentration range, approaching 30 μM trimethoprim, may provide maximal folate stress while still maintaining subinhibitory characteristics in most bacterial strains. Concentrations at or near 30 μM may approach the threshold where trimethoprim alone may begin to exhibit measurable growth inhibition, potentially transitioning from subinhibitory to minimally inhibitory concentrations. The 30 μM concentration may be suitable for treating severe infections or antibiotic-resistant strains where maximal synthetic lethal effects may be needed.

The concentration range between 5 μM and 30 μM may accommodate individual patient variations in drug metabolism, renal clearance, and infection characteristics. Patients with impaired renal function may require lower trimethoprim concentrations to avoid accumulation and potential toxicity, while patients with severe infections may benefit from higher concentrations within the therapeutic range. The flexibility of the concentration range may allow for personalized dosing approaches that optimize antimicrobial efficacy while minimizing adverse effects.

The subinhibitory nature of the trimethoprim concentration range may be maintained through careful monitoring of bacterial growth responses and adjustment of dosing protocols based on observed effects. The concentration may be considered subinhibitory when bacterial cultures treated with trimethoprim alone maintain growth rates and final optical density measurements comparable to untreated control cultures. This subinhibitory characteristic may be essential for the synthetic lethal mechanism, as higher concentrations that cause direct growth inhibition may mask or interfere with the conditional essentiality of FolE2.

The method may involve dose escalation within the 5 μM to 30 μM range based on treatment response and patient tolerance. Initial treatment may begin with lower concentrations such as 10 μM to 15 μM trimethoprim, with subsequent increases to 20 μM or higher concentrations if enhanced antimicrobial effects may be needed. The dose escalation approach may allow for optimization of the synthetic lethal interaction while monitoring for any signs of direct antimicrobial activity that might indicate transition beyond subinhibitory concentrations.

The trimethoprim concentration may be adjusted based on the specific FolE2 inhibitor used in the combination therapy. Different inhibitor compounds may exhibit varying degrees of potency enhancement as trimethoprim concentrations increase, potentially requiring different optimal trimethoprim concentrations for maximal synthetic lethal effects. The 20 μM trimethoprim concentration may provide effective enhancement for most FolE2 inhibitors, but specific combinations may benefit from fine-tuning within the broader 5 μM to 30 μM range.

The maintenance of subinhibitory concentrations may be verified through growth curve analysis and optical density measurements that confirm bacterial viability in the presence of trimethoprim alone. The subinhibitory nature may be characterized by growth curves that show minimal deviation from control conditions, with final optical density measurements remaining within normal ranges for bacterial cultures. This verification may ensure that the synthetic lethal mechanism operates through conditional essentiality rather than through direct antimicrobial effects of trimethoprim.

The method for treating melioidosis may utilize dehydrocostus lactone as the FolE2 enzyme inhibitor, where the dehydrocostus lactone may have an IC₅₀ value of between 1.7 μM and 2.5 μM in the presence of trimethoprim. This specific potency range may provide a therapeutic window that enables precise dosing strategies for clinical applications while maintaining the synthetic lethal interaction that characterizes the treatment approach. The IC₅₀ values within this range may indicate that dehydrocostus lactone achieves effective antimicrobial activity at low micromolar concentrations when combined with appropriate trimethoprim levels.

The IC₅₀ value of 1.7 μM may represent the lower bound of the potency range, which may be observed under optimal conditions where trimethoprim concentrations create maximal folate stress and render FolE2 maximally essential for bacterial survival. At this potency level, dehydrocostus lactone may achieve 50% growth inhibition at concentrations that may minimize potential off-target effects while maintaining effective antimicrobial activity against Burkholderia pseudomallei. The 1.7 μM IC₅₀ value may be particularly suitable for patients who may require lower drug exposures due to sensitivity concerns or concurrent medications.

The IC₅₀ value of 2.5 μM may represent the upper bound of the potency range, which may be observed under conditions where trimethoprim concentrations provide sufficient but not maximal folate stress for inducing FolE2 conditional essentiality. At this potency level, dehydrocostus lactone may still achieve effective growth inhibition while providing a margin of safety that may accommodate variations in patient response or drug metabolism. The 2.5 μM IC₅₀ value may be suitable for standard dosing protocols where consistent antimicrobial effects may be needed across diverse patient populations.

The potency range between 1.7 μM and 2.5 μM may enable precise dosing strategies by providing predictable concentration-response relationships that may guide clinical dose selection. The narrow range may indicate consistent inhibitory activity that may allow clinicians to calculate appropriate dosing regimens based on pharmacokinetic parameters and desired therapeutic outcomes. The predictable potency may facilitate dose optimization approaches that may balance antimicrobial efficacy against potential adverse effects.

The method may utilize dehydrocostus lactone concentrations that correspond to the IC₅₀ range for achieving therapeutic effects in clinical applications. Dosing strategies may target plasma concentrations that exceed the IC₅₀ values by appropriate margins to ensure effective antimicrobial activity while accounting for factors such as protein binding, tissue distribution, and drug clearance. The specific IC₅₀ range may provide guidance for determining minimum effective concentrations and maximum tolerated doses in clinical protocols.

The IC₅₀ values between 1.7 μM and 2.5 μM may reflect the mechanism-based inhibition characteristics of dehydrocostus lactone, where the compound may achieve potent enzyme inactivation through covalent modification of the catalytic cysteine residue. The consistent potency range may indicate that the covalent inhibition mechanism provides reliable and reproducible antimicrobial effects that may be suitable for therapeutic applications. The mechanism-based nature of the inhibition may contribute to the sustained antimicrobial activity observed with dehydrocostus lactone treatment.

The method may involve dose escalation strategies that may begin with concentrations corresponding to the lower end of the IC₅₀ range and may increase to higher concentrations based on treatment response. Initial dosing may target concentrations that achieve the 1.7 μM IC₅₀ level, with subsequent increases toward the 2.5 μM range if enhanced antimicrobial effects may be needed. This escalation approach may allow for optimization of therapeutic outcomes while minimizing the risk of adverse effects associated with higher drug concentrations.

The precise dosing strategies enabled by the IC₅₀ range may accommodate individual patient factors that may influence drug response and tolerance. Patients with impaired hepatic or renal function may benefit from dosing approaches that target the lower end of the IC₅₀ range to avoid drug accumulation, while patients with severe infections may require dosing that achieves concentrations corresponding to the upper end of the range. The defined potency range may provide flexibility for personalized treatment approaches.

The method may utilize therapeutic drug monitoring approaches that may measure dehydrocostus lactone concentrations in patient samples and may adjust dosing to maintain levels within the effective range corresponding to the IC₅₀ values. The specific potency range may provide target concentrations for monitoring protocols that may ensure adequate drug exposure while avoiding potentially toxic levels. The monitoring approach may be particularly valuable during the intensive therapy phase where rapid antimicrobial effects may be needed.

The IC₅₀ values between 1.7 μM and 2.5 μM may enable combination dosing strategies that may coordinate dehydrocostus lactone concentrations with trimethoprim levels to optimize the synthetic lethal interaction. The method may involve simultaneous adjustment of both components to maintain the appropriate concentration ratios that may maximize antimicrobial efficacy. The defined potency range for dehydrocostus lactone may provide a reference point for calculating optimal combination ratios with various trimethoprim concentrations.

The clinical application of dehydrocostus lactone at concentrations corresponding to the IC₅₀ range may provide advantages in terms of treatment duration and dosing frequency. The potent antimicrobial activity achieved within the 1.7 μM to 2.5 μM range may allow for less frequent dosing or shorter treatment courses compared to less potent alternatives. The efficient antimicrobial activity may reduce the overall drug burden on patients while maintaining effective therapeutic outcomes.

The method may incorporate pharmacokinetic modeling approaches that may predict dehydrocostus lactone concentrations based on dosing regimens and may ensure that therapeutic levels corresponding to the IC₅₀ range may be achieved and maintained throughout the treatment course. The modeling may account for factors such as absorption, distribution, metabolism, and elimination to optimize dosing strategies for individual patients. The specific IC₅₀ range may provide target parameters for the pharmacokinetic models.

The precise dosing strategies may be particularly valuable for treating melioidosis infections in resource-limited settings where drug availability may be constrained and treatment optimization may be essential. The defined IC₅₀ range may enable efficient use of available drug supplies by providing clear guidance on minimum effective concentrations. The predictable potency may reduce the need for extensive dose-finding studies in clinical applications, potentially accelerating treatment implementation in endemic areas.

The method may utilize the IC₅₀ range to establish dosing protocols for different phases of melioidosis treatment, with intensive therapy potentially requiring concentrations toward the upper end of the range and eradication therapy potentially utilizing concentrations toward the lower end. The flexibility provided by the potency range may allow for treatment protocols that may be tailored to the specific requirements of each treatment phase while maintaining consistent antimicrobial pressure against the pathogenic bacteria.

In the method for treating melioidosis, the FolE2 enzyme inhibitor may act as a mechanism-based inhibitor that covalently modifies a catalytic cysteine residue of the FolE2 enzyme. Mechanism-based inhibitors may undergo enzyme-catalyzed reactions that result in the formation of covalent bonds with amino acid residues in the active site, leading to irreversible enzyme inactivation. In some cases, the FolE2 enzyme inhibitor forms a covalent bond through Michael addition reactions with nucleophilic amino acid residues.

The catalytic cysteine residue may be Cys154. Cys154 may serve as a nucleophile that attacks electrophilic centers present in the FolE2 enzyme inhibitor molecules. In some cases, compounds containing exocyclic enone moieties, such as dehydrocostus lactone and parthenolide, may undergo Michael addition reactions with the sulfur atom of Cys154. The resulting covalent modification may displace the metal ion from the active site and may cause structural disruption that renders the enzyme inactive.

The mechanism-based inhibition may provide sustained antimicrobial effects in the treatment method. In some cases, the irreversible nature of the covalent modification may result in prolonged enzyme inactivation that persists beyond the clearance of the inhibitor compound from the system. The sustained enzyme inactivation may maintain the chemical synthetic lethal effect with subinhibitory trimethoprim concentrations for extended periods.

The irreversible enzyme inactivation may enable reduced dosing frequency requirements in the treatment method. In some cases, the prolonged enzyme inactivation may allow for less frequent administration of the pharmaceutical composition while maintaining therapeutic efficacy against Burkholderia pseudomallei. The reduced dosing frequency may improve patient compliance and may reduce the potential for adverse effects associated with frequent dosing regimens.

The combination in the treatment method may exhibit greater than 90% growth suppression against Burkholderia pseudomallei while having minimal effect on commensal bacteria. The selective antimicrobial activity may provide therapeutic advantages by preserving beneficial microorganisms that contribute to host health and microbiome stability. In some cases, the preservation of commensal bacteria may reduce the risk of secondary infections and may maintain normal physiological functions associated with a healthy microbiome.

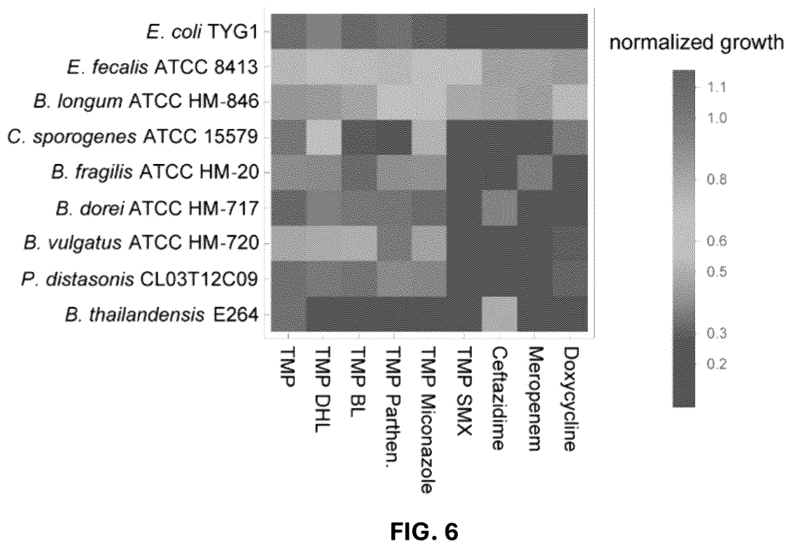

Referring to FIG. 6, the selectivity profile of the combination treatment may be demonstrated through comparative growth inhibition studies across multiple bacterial species. The heatmap shows normalized growth values for various bacterial strains under different treatment conditions, with values ranging from approximately 0.2 to 1.1 on the color scale. The data may demonstrate differential effects of the combination treatment on pathogenic versus commensal bacterial species.

The commensal bacteria may include Bacteroides fragilis, Bifidobacterium longum, Clostridium sporogenes, and Parabacteroides distasonis. These bacterial species may represent important components of the human gut microbiome that contribute to digestive health, immune function, and metabolic processes. In some cases, the preservation of these commensal bacteria during antimicrobial treatment may maintain microbiome diversity and may prevent dysbiosis-related complications.

As shown in FIG. 6, the combination treatment conditions may demonstrate selective growth inhibition patterns. The treatment combinations involving trimethoprim with dehydrocostus lactone (TMP_DHL), trimethoprim with β-lapachone (TMP_BL), and trimethoprim with parthenolide (TMP_Parthen.) may show differential effects on Burkholderia thailandensis compared to the commensal bacterial strains. The selectivity data may indicate that the combination treatments maintain higher normalized growth values for commensal species while achieving substantial growth suppression against the target pathogen.

The minimal effect on commensal bacteria may result from the absence of FolE2 enzyme in many commensal bacterial species. FolE2 may be present in only approximately 20% of microbial genomes, and many beneficial gut bacteria may lack this enzyme entirely. In some cases, the absence of the target enzyme in commensal bacteria may render these organisms insensitive to FolE2 inhibitors, thereby providing a mechanism for selective antimicrobial activity.

The selectivity profile may extend to additional commensal bacterial species including Bacteroides dorei, Bacteroides vulgatus, and Enterococcus faecalis. These bacterial species may also demonstrate resistance to the combination treatment due to the absence of FolE2 enzyme or alternative metabolic pathways that do not rely on FolE2 activity. In some cases, the broad selectivity against multiple commensal species may provide comprehensive microbiome preservation during treatment.

With continued reference to FIG. 6, the comparison with conventional antimicrobial treatments may highlight the selectivity advantages of the combination approach. Standard antimicrobial agents such as trimethoprim-sulfamethoxazole (TMP_SMX), ceftazidime, meropenem, and doxycycline may show broader antimicrobial activity that affects both pathogenic and commensal bacterial species. The reduced selectivity of conventional treatments may result in more extensive microbiome disruption compared to the combination treatment method.

The preservation of beneficial microorganisms may provide clinical advantages in the treatment of melioidosis. In some cases, maintaining a healthy microbiome during antimicrobial therapy may reduce the risk of opportunistic infections, may support immune function, and may facilitate faster recovery. The selective antimicrobial activity may also reduce the likelihood of antibiotic-associated complications such as Clostridioides difficile infections that may occur following broad-spectrum antimicrobial treatment.

A method for identifying antimicrobial drug targets may comprise screening a library of compounds for growth inhibition of a target bacterial pathogen in the presence of a subinhibitory concentration of a known antibiotic. The screening method may utilize high-throughput approaches that enable evaluation of large compound collections to identify molecules that exhibit chemical synthetic lethality when combined with established antimicrobial agents. The target bacterial pathogen may be selected based on clinical relevance, antibiotic resistance patterns, or specific metabolic characteristics that may render the organism susceptible to synthetic lethal interactions.

The library of compounds may comprise structurally diverse molecules including natural products and FDA-approved small-molecule drugs. Natural products may provide chemical scaffolds that have been evolutionarily optimized for biological activity and may offer novel mechanisms of action against bacterial targets. FDA-approved drugs may provide opportunities for drug repurposing applications where compounds developed for other therapeutic indications may exhibit antimicrobial activity through synthetic lethal mechanisms. The combination of natural products and approved drugs may provide a comprehensive chemical space for identifying synthetic lethal interactions.

The screening method may utilize different assay formats including 96-well plates, flask cultures, and 384-well plates for compound validation and kinetic studies. The 96-well plate format may provide a standardized approach for initial compound screening that enables parallel evaluation of multiple compounds under controlled conditions. Flask cultures may provide more physiologically relevant growth conditions that may better reflect bacterial behavior in clinical infections, allowing for validation of hits identified in plate-based assays.

Referring to FIG. 2, the screening approach may involve systematic evaluation of compound libraries against bacterial cultures grown in the presence and absence of subinhibitory concentrations of known antibiotics. The scatter plot demonstrates the screening results across approximately 1320 compounds, with library compound numbers plotted against Z-score values that quantify growth inhibition effects. The screening data may show that most compounds cluster near zero Z-score values, indicating minimal effects on bacterial growth under the tested conditions.

The screening methodology may involve parallel testing of each compound under two conditions: with and without the subinhibitory concentration of the known antibiotic. Compounds may be evaluated at standardized concentrations across the library to enable direct comparison of growth inhibition effects. The parallel testing approach may allow for identification of compounds that exhibit differential activity depending on the presence of the known antibiotic, indicating potential synthetic lethal interactions.

Growth inhibition may be measured by optical density at 600 nm after incubation for 24 to 48 hours. The optical density measurements may provide quantitative assessment of bacterial growth that may be standardized across different experimental conditions and compound treatments. The 24 to 48 hour incubation period may allow sufficient time for bacterial growth while enabling detection of growth inhibition effects that may develop over extended time courses.

The method may involve identifying compounds that exhibit no growth inhibition individually but cause growth inhibition when combined with the subinhibitory concentration of the known antibiotic. The identification process may utilize statistical analysis approaches that define growth inhibition thresholds based on standard deviations from control conditions. Compounds that reduce growth by two standard deviations or more in the presence of the known antibiotic, while showing minimal effects in its absence, may be classified as synthetic lethal hits.

With continued reference to FIG. 2, the screening results may identify multiple compounds that meet the synthetic lethal criteria, as demonstrated by data points that extend below the threshold line in the presence of trimethoprim while remaining near baseline levels in its absence. The chemical structures of validated inhibitor compounds may represent diverse molecular scaffolds including sesquiterpene lactones, quinones, azole antifungals, and other pharmacologically active molecules. This structural diversity may indicate that multiple chemical approaches may be effective for achieving synthetic lethal interactions.

The 384-well plate format may be utilized for detailed kinetic studies and dose-response analysis of identified hit compounds. The higher density format may enable more extensive concentration-response studies that may characterize the potency and efficacy of synthetic lethal interactions. The 384-well format may also facilitate enzymatic assays for target identification and validation studies that may require multiple substrate concentrations and inhibitor dilutions.

Flask culture validation may provide confirmation of synthetic lethal effects under conditions that more closely approximate physiological bacterial growth. The flask format may allow for larger culture volumes that may reduce variability associated with edge effects and evaporation that may occur in plate-based assays. Flask cultures may also enable extended time course studies that may characterize the kinetics of synthetic lethal interactions over longer incubation periods.

The screening method may incorporate positive and negative control conditions to ensure assay reliability and enable normalization of growth inhibition data. Positive controls may include known antimicrobial agents or genetic mutants that exhibit growth defects under the screening conditions. Negative controls may include vehicle treatments and wild-type bacterial strains that demonstrate normal growth patterns. The control conditions may provide reference points for interpreting compound effects and may enable quality control assessment of screening performance.

The method may involve determining the molecular target of the identified compounds through biochemical and genetic approaches. Target identification may utilize enzymatic assays that test compound effects against purified proteins involved in bacterial metabolism. Genetic approaches may involve complementation studies using bacterial strains with defined gene deletions or overexpression constructs that may reveal the specific pathways affected by the identified compounds.

Enzymatic assays may be conducted using purified target proteins to confirm direct inhibition by the identified compounds. The assays may measure enzyme activity in the presence and absence of potential inhibitors to establish concentration-response relationships and inhibition mechanisms. Kinetic analysis may determine whether compounds function as competitive, noncompetitive, or uncompetitive inhibitors, providing insights into binding modes and mechanisms of action.

The method may involve validating the molecular target as a conditionally essential enzyme that becomes essential in the presence of the subinhibitory concentration of the known antibiotic. Validation studies may utilize genetic approaches including gene deletion mutants and complementation experiments to confirm that the identified target enzyme may be dispensable under normal growth conditions but may become essential under the stress conditions created by the known antibiotic.

Complementation experiments may involve introducing the target gene on plasmid vectors into deletion mutant strains to restore normal growth patterns. The restoration of growth through complementation may confirm that the synthetic lethal phenotype results from loss of the specific target enzyme rather than from secondary effects of the gene deletion. The complementation approach may provide definitive validation that the identified enzyme serves as the molecular target for the synthetic lethal interaction.

The validation process may also involve testing the synthetic lethal interaction across multiple bacterial strains and species to establish the breadth of the antimicrobial approach. Strains that possess the target enzyme may exhibit synthetic lethal phenotypes when treated with the compound and antibiotic combination, while strains lacking the target may remain unaffected. This differential susceptibility pattern may provide additional validation of the target identification and may indicate the potential selectivity of the antimicrobial approach.

The method for identifying antimicrobial drug targets may provide a systematic approach for discovering new therapeutic targets and developing novel antimicrobial strategies. The synthetic lethal screening approach may identify metabolic vulnerabilities that may not be apparent through conventional antimicrobial screening methods. The identification of conditionally essential enzymes may expand the range of potential drug targets beyond those that may be essential under normal growth conditions.

The screening methodology may be applicable to various bacterial pathogens and may be adapted for different known antibiotics to identify diverse synthetic lethal interactions. The flexibility of the approach may enable discovery of pathogen-specific vulnerabilities that may provide selective antimicrobial strategies. The method may also facilitate identification of combination therapies that may overcome antibiotic resistance mechanisms by targeting alternative metabolic pathways.

The compound library composition may comprise natural products and FDA-approved small-molecule drugs that provide a comprehensive chemical space for identifying synthetic lethal interactions. Natural products may represent molecules that have been evolutionarily optimized for biological activity through millions of years of natural selection processes. These compounds may exhibit complex three-dimensional structures and diverse functional groups that may interact with biological targets in ways that may not be readily accessible through synthetic chemistry approaches.

Natural products in the compound library may include secondary metabolites derived from plants, fungi, bacteria, and marine organisms. Plant-derived natural products may comprise alkaloids, terpenoids, phenolic compounds, and glycosides that may exhibit antimicrobial, anti-inflammatory, or cytotoxic activities. Fungal metabolites may include polyketides, peptides, and hybrid biosynthetic products that may demonstrate novel mechanisms of action against bacterial targets. Bacterial natural products may encompass antibiotics, siderophores, and signaling molecules that may interfere with bacterial metabolism or communication pathways.

The structural diversity of natural products may provide access to chemical scaffolds that may not be represented in synthetic compound libraries. Natural products may contain unusual ring systems, stereochemical arrangements, and functional group combinations that may result from complex biosynthetic pathways. This structural complexity may enable interactions with protein targets that may require specific three-dimensional complementarity or may involve multiple binding sites simultaneously.

FDA-approved small-molecule drugs may represent compounds that have undergone extensive clinical testing and regulatory approval for various therapeutic indications. These drugs may provide opportunities for repurposing applications where compounds developed for treating human diseases may exhibit antimicrobial activity through mechanisms that may differ from their primary therapeutic targets. The repurposing approach may leverage existing safety data, pharmacokinetic profiles, and manufacturing processes that may accelerate development timelines for new antimicrobial applications.

The FDA-approved drug component of the library may include compounds from diverse therapeutic categories including cardiovascular agents, neurological medications, anti-inflammatory drugs, antifungal agents, and antiviral compounds. Cardiovascular drugs may include calcium channel blockers, ACE inhibitors, and beta-blockers that may exhibit off-target effects against bacterial enzymes or membrane systems. Neurological medications may comprise antipsychotics, antidepressants, and anticonvulsants that may interfere with bacterial signaling pathways or metabolic processes.

Anti-inflammatory drugs in the library may include nonsteroidal anti-inflammatory drugs, corticosteroids, and immunosuppressive agents that may exhibit antimicrobial properties through inhibition of bacterial enzymes or disruption of cellular processes. Antifungal agents may demonstrate cross-kingdom activity against bacterial targets, particularly when bacterial metabolism may share similarities with fungal biochemical pathways. Antiviral compounds may interfere with bacterial replication or transcription processes that may share mechanistic features with viral life cycles.

The combination of natural products and FDA-approved drugs in the compound library may provide complementary advantages for synthetic lethal screening applications. Natural products may offer novel chemical scaffolds and mechanisms of action that may not be represented in synthetic drug libraries, while FDA-approved drugs may provide compounds with established safety profiles and known pharmacological properties. The dual composition may maximize the probability of identifying effective synthetic lethal combinations while minimizing development risks associated with unknown compounds.

The use of established compound libraries may accelerate drug discovery by leveraging existing safety and pharmacological data that may reduce development timelines and regulatory requirements. Natural product libraries may provide access to compounds with documented biological activities and established extraction or synthesis methods that may facilitate scale-up for therapeutic applications. The historical use of many natural products in traditional medicine may provide preliminary safety information that may support clinical development efforts.

FDA-approved drugs may offer significant advantages in terms of regulatory pathways for repurposing applications. These compounds may have established manufacturing processes, quality control standards, and distribution networks that may enable rapid clinical implementation once antimicrobial efficacy may be demonstrated. The existing regulatory approval for other indications may provide a foundation for expedited review processes that may focus on antimicrobial efficacy rather than comprehensive safety evaluation.

The pharmacokinetic data available for FDA-approved drugs may enable predictive modeling of drug concentrations and tissue distribution patterns that may be relevant for antimicrobial applications. Absorption, distribution, metabolism, and excretion profiles may be well-characterized for these compounds, allowing for rational dose selection and treatment protocol development. The availability of pharmacokinetic data may reduce the need for extensive preclinical studies that may otherwise be required for novel compounds.

Drug-drug interaction profiles may be established for FDA-approved compounds, providing guidance for combination therapy approaches and contraindication assessment. The interaction data may enable identification of compounds that may be safely combined with existing antimicrobial agents or that may require dose adjustments in patients receiving concurrent medications. The established interaction profiles may facilitate clinical trial design and patient safety monitoring protocols.

Manufacturing and formulation technologies may be well-developed for FDA-approved drugs, enabling rapid production scale-up for clinical applications. Established synthetic routes, purification methods, and quality control procedures may reduce the time and cost associated with drug development compared to novel compounds that may require extensive process development. The availability of multiple generic manufacturers for many FDA-approved drugs may provide supply chain redundancy and cost advantages for large-scale therapeutic applications.

The regulatory pathway for drug repurposing may involve abbreviated development timelines compared to novel drug development programs. Regulatory agencies may provide expedited review processes for repurposing applications that may focus on demonstrating efficacy for the new indication while relying on existing safety data. The abbreviated pathway may reduce development costs and may enable faster patient access to new antimicrobial therapies.

Clinical trial design for repurposed drugs may benefit from established dosing regimens, safety monitoring protocols, and adverse event profiles that may inform study protocols and patient selection criteria. The availability of clinical experience with these compounds may enable more efficient trial design and may reduce the risk of unexpected safety issues during clinical development. The established clinical experience may also facilitate investigator familiarity and patient acceptance of repurposed therapies.

The compound library composition may enable systematic exploration of chemical space that encompasses both evolutionarily optimized natural products and clinically validated synthetic compounds. The comprehensive coverage may increase the probability of identifying effective synthetic lethal combinations while providing multiple options for therapeutic development. The diversity of the library may also enable structure-activity relationship studies that may guide optimization of lead compounds for enhanced potency or selectivity.

Quality control standards for both natural products and FDA-approved drugs may ensure consistent compound identity and purity across screening campaigns. Natural product libraries may utilize standardized extraction procedures, analytical characterization methods, and storage conditions that may maintain compound stability and biological activity. FDA-approved drug libraries may benefit from pharmaceutical-grade compound sources that may provide high purity and consistent quality for screening applications.

The scalability of compound libraries comprising natural products and FDA-approved drugs may support expansion of screening efforts to additional bacterial pathogens or alternative synthetic lethal combinations. Natural product sources may provide renewable supplies of bioactive compounds through cultivation or fermentation approaches, while FDA-approved drugs may be available through established commercial sources. The scalable nature of the library may enable comprehensive screening programs that may identify multiple therapeutic targets and combination strategies.

The method for identifying antimicrobial drug targets may utilize optical density measurements at 600 nm as a standardized approach for quantifying bacterial growth and assessing antimicrobial activity. Optical density at 600 nm may provide a wavelength that minimizes interference from culture media components while providing sensitive detection of bacterial cell density changes that occur during growth inhibition. The 600 nm wavelength may be selected because bacterial cells exhibit minimal light absorption at this wavelength, allowing optical density changes to primarily reflect alterations in light scattering caused by changes in cell number and biomass.

The optical density measurement method may involve spectrophotometric analysis of bacterial cultures using microplate readers or spectrophotometers equipped with 600 nm filters or monochromators. The measurement approach may utilize standardized culture volumes and optical path lengths to ensure reproducible quantification across different experimental conditions and compound treatments. The optical density values may be recorded as absorbance units that correlate directly with bacterial cell density and total biomass in the culture samples.

Incubation periods of 24 to 48 hours may provide sufficient time for bacterial growth while enabling detection of growth inhibition effects that may develop over extended time courses. The 24-hour incubation period may be suitable for rapidly growing bacterial species that achieve stationary phase growth within this timeframe, allowing for assessment of both growth rate effects and final biomass accumulation. The 48-hour incubation period may accommodate slower-growing bacterial species or may enable detection of delayed growth inhibition effects that may not be apparent at earlier time points.

The standardized measurement approach may involve baseline optical density measurements taken at the initiation of incubation to establish starting cell densities across all experimental conditions. The baseline measurements may enable calculation of growth fold-changes and may provide normalization factors that account for variations in initial inoculum density. The standardized starting conditions may ensure that growth inhibition effects reflect compound activity rather than differences in initial bacterial load.

Multiple time point measurements may be conducted throughout the 24 to 48 hour incubation period to generate growth curves that characterize the kinetics of growth inhibition. The time course measurements may reveal whether compounds exhibit immediate growth inhibition effects or whether inhibitory activity develops gradually over extended incubation periods. The kinetic data may provide insights into mechanism of action and may enable differentiation between bacteriostatic and bactericidal effects.

The optical density measurement method may incorporate appropriate controls including vehicle-treated cultures, untreated cultures, and positive control compounds with known antimicrobial activity. The control conditions may provide reference points for interpreting compound effects and may enable quality control assessment of assay performance. The vehicle controls may account for potential solvent effects on bacterial growth, while positive controls may confirm assay sensitivity and dynamic range.

Standardized culture conditions may be maintained throughout the incubation period including consistent temperature, atmospheric composition, and agitation parameters that optimize bacterial growth. The controlled environmental conditions may minimize variability in growth measurements and may ensure reproducible assessment of compound effects across different experimental sessions. The standardized conditions may also enable comparison of results across different bacterial species and compound libraries.

The quantitative nature of optical density measurements may enable calculation of specific growth inhibition parameters including IC₅₀ values, minimal inhibitory concentrations, and growth rate constants. The quantitative data may facilitate statistical analysis approaches that determine significance of growth inhibition effects and may enable ranking of compound potency across large screening libraries. The numerical data may also support dose-response modeling that characterizes concentration-activity relationships for identified hit compounds.

Reproducibility of the optical density measurement approach may be enhanced through standardized protocols that specify culture media composition, inoculation procedures, and measurement timing. The standardized protocols may reduce inter-assay variability and may enable consistent results across different laboratory settings and personnel. The reproducible nature of the measurements may support regulatory submissions and clinical development activities that require validated analytical methods.

The optical density measurement method may accommodate high-throughput screening applications through automation of culture handling, incubation, and measurement procedures. Automated systems may enable parallel processing of large compound libraries while maintaining consistent measurement conditions and reducing manual handling errors. The high-throughput compatibility may accelerate drug discovery timelines and may enable comprehensive screening of diverse chemical libraries.

Quality control measures may be implemented to ensure measurement accuracy including regular calibration of spectrophotometric equipment, verification of wavelength accuracy, and assessment of optical path length consistency. The quality control procedures may maintain measurement precision and may enable detection of systematic errors that could affect screening results. The validated measurement approach may provide confidence in compound activity assessments and may support regulatory requirements for analytical method validation.

The optical density measurement approach may enable real-time monitoring of bacterial growth through kinetic measurement protocols that record optical density changes at regular intervals throughout the incubation period. The real-time monitoring may provide detailed growth curves that reveal the onset and progression of growth inhibition effects. The kinetic data may enable more sensitive detection of subtle growth effects that might not be apparent in endpoint measurements alone.

Data analysis procedures for optical density measurements may involve normalization approaches that account for background absorbance, media interference, and plate-to-plate variations. The normalization methods may improve data quality and may enable accurate comparison of compound effects across different experimental conditions. The standardized data analysis may facilitate identification of genuine growth inhibition effects while minimizing false positive results from measurement artifacts.

The optical density measurement method may provide advantages over alternative growth assessment approaches including colony counting, biomass determination, and metabolic activity assays. The optical density approach may offer rapid, non-destructive measurement that preserves culture samples for additional analyses while providing quantitative data suitable for statistical evaluation. The method may also demonstrate superior throughput compared to labor-intensive colony counting procedures while maintaining comparable accuracy for growth assessment applications.

The method for identifying antimicrobial drug targets may identify compounds that function as mechanism-based inhibitors that covalently modify a catalytic amino acid residue. Mechanism-based inhibitors may represent a specialized class of enzyme inhibitors that undergo enzyme-catalyzed reactions to form covalent bonds with amino acid residues in the active site, resulting in irreversible enzyme inactivation. These inhibitors may be distinguished from reversible inhibitors by their ability to form permanent chemical bonds with target enzymes, leading to sustained inhibition that persists beyond clearance of the inhibitor molecule from the biological system.

The mechanism-based inhibition process may involve initial binding of the inhibitor compound to the enzyme active site, followed by enzyme-catalyzed conversion of the inhibitor into a reactive intermediate that subsequently forms covalent bonds with nucleophilic amino acid residues. The catalytic amino acid residue may serve as both a participant in the normal enzymatic reaction and as a target for covalent modification by the mechanism-based inhibitor. This dual role may enable selective targeting of the catalytic residue while minimizing off-target effects on non-catalytic amino acids.

Covalent modification of the catalytic amino acid residue may occur through various chemical mechanisms including alkylation, acylation, or addition reactions that result in permanent chemical bond formation between the inhibitor and the amino acid sidechain. The specific mechanism may depend on the chemical structure of the inhibitor compound and the nature of the catalytic residue. Nucleophilic amino acids such as cysteine, serine, and lysine may serve as common targets for covalent modification due to their reactive sidechains that may participate in nucleophilic attack reactions.

The identification of mechanism-based inhibitors through the screening method may involve recognition of compounds that exhibit time-dependent inhibition kinetics, where enzyme activity decreases progressively during incubation with the inhibitor. Time-dependent inhibition may indicate that the inhibitor undergoes a chemical reaction with the enzyme that results in progressive accumulation of covalently modified enzyme molecules. This kinetic signature may distinguish mechanism-based inhibitors from competitive or noncompetitive reversible inhibitors that exhibit immediate equilibrium binding.

Mechanism-based inhibitors may demonstrate enhanced selectivity compared to reversible inhibitors because the covalent modification process may require specific structural complementarity between the inhibitor and the enzyme active site. The enzyme-catalyzed activation of the inhibitor may ensure that reactive intermediates are generated only in the presence of the target enzyme, reducing the likelihood of non-specific reactions with other cellular components. This selectivity mechanism may minimize off-target effects and may reduce toxicity associated with indiscriminate protein modification.

The sustained antimicrobial activity provided by mechanism-based inhibitors may result from the irreversible nature of the covalent modification, which may prevent enzyme recovery through inhibitor dissociation. Once the catalytic amino acid residue has been covalently modified, the enzyme may remain inactive until new enzyme molecules are synthesized through protein translation processes. The sustained inhibition may maintain antimicrobial pressure against bacterial pathogens for extended periods, potentially enabling less frequent dosing regimens and improved patient compliance.

The reduced potential for rapid resistance development may represent a significant advantage of mechanism-based inhibitors compared to reversible inhibitors. Bacterial resistance to reversible inhibitors may develop through mutations that reduce inhibitor binding affinity or through upregulation of target enzyme expression that overcomes competitive inhibition. Mechanism-based inhibitors may be less susceptible to these resistance mechanisms because the covalent modification process may be less sensitive to minor changes in binding affinity, and enzyme overexpression may not overcome irreversible inactivation.

Resistance to mechanism-based inhibitors may require more substantial genetic changes such as mutations that alter the catalytic mechanism to eliminate the reactive amino acid residue or that provide alternative metabolic pathways that bypass the inhibited enzyme. These types of resistance mutations may be less likely to occur spontaneously and may impose greater fitness costs on bacterial pathogens compared to the minor binding site mutations that may confer resistance to reversible inhibitors. The higher genetic barrier to resistance may extend the useful therapeutic lifetime of mechanism-based inhibitors.

The screening method may identify mechanism-based inhibitors through biochemical assays that demonstrate covalent enzyme modification, including mass spectrometry analysis that reveals molecular weight increases corresponding to inhibitor attachment, or through dialysis experiments that show persistent enzyme inhibition after removal of free inhibitor molecules. These analytical approaches may confirm that the identified compounds function through covalent mechanisms rather than through reversible binding interactions.

The catalytic amino acid residue targeted by mechanism-based inhibitors may vary depending on the specific enzyme and inhibitor combination identified through the screening process. Cysteine residues may serve as common targets due to their nucleophilic sulfur atoms that may readily participate in alkylation or addition reactions with electrophilic inhibitor compounds. Serine residues may be targeted by inhibitors that form acyl-enzyme intermediates, while lysine residues may be modified by compounds that react with amino groups.

The chemical reactivity of the catalytic amino acid residue may be enhanced by the enzyme active site environment, which may lower the pKa of ionizable groups or may position reactive residues in conformations that favor nucleophilic attack. The enzyme active site may also provide binding interactions that orient the inhibitor compound appropriately for reaction with the catalytic residue while excluding water molecules that might compete for reaction with the electrophilic center.

Mechanism-based inhibitors identified through the screening method may include compounds containing reactive functional groups such as α,β-unsaturated carbonyls, epoxides, halomethyl ketones, or other electrophilic moieties that may undergo nucleophilic attack by amino acid sidechains. The reactive groups may be masked or activated through enzyme-catalyzed reactions that generate electrophilic intermediates capable of covalent bond formation. The enzyme-mediated activation may ensure that reactive species are generated only at the target site, minimizing non-specific reactions.

The irreversible nature of mechanism-based inhibition may provide advantages in treating chronic or persistent bacterial infections where sustained antimicrobial pressure may be needed to achieve complete bacterial clearance. The prolonged enzyme inactivation may prevent bacterial recovery during treatment interruptions and may reduce the likelihood of infection relapse. The sustained activity may be particularly valuable for treating intracellular pathogens or biofilm-associated infections where drug penetration may be limited and sustained local concentrations may be difficult to maintain.

The development of mechanism-based inhibitors as antimicrobial agents may benefit from structure-activity relationship studies that optimize the balance between reactivity and selectivity. The inhibitor compounds may require sufficient electrophilicity to react with the target amino acid residue while maintaining selectivity for the intended enzyme target. The optimization process may involve modification of reactive groups, adjustment of leaving group properties, or incorporation of recognition elements that enhance binding specificity.

The screening method may identify mechanism-based inhibitors that target different classes of enzymes involved in bacterial metabolism, including enzymes in folate biosynthesis, cell wall synthesis, DNA replication, or energy metabolism. The diversity of potential targets may provide multiple opportunities for developing mechanism-based antimicrobial agents that may address different aspects of bacterial physiology. The identification of multiple mechanism-based inhibitors may also enable combination therapy approaches that target multiple pathways simultaneously.

The validation of mechanism-based inhibitors identified through the screening method may involve kinetic studies that demonstrate time-dependent inhibition, protection experiments that show substrate or cofactor protection against inactivation, and partition ratio determinations that quantify the efficiency of the inactivation process. These studies may confirm the mechanism-based nature of the inhibition and may provide parameters for optimizing inhibitor design and dosing strategies.

The clinical development of mechanism-based inhibitors may require consideration of the irreversible nature of the enzyme modification, which may influence dosing strategies, safety assessment, and monitoring protocols. The sustained enzyme inhibition may enable reduced dosing frequencies but may also require careful dose selection to avoid excessive enzyme inactivation that could lead to toxicity. The irreversible mechanism may also influence the design of clinical trials and the interpretation of pharmacokinetic and pharmacodynamic relationships.

The method for identifying antimicrobial drug targets may identify mechanism-based inhibitors that comprise sesquiterpene lactones with exocyclic enone moieties. Sesquiterpene lactones may represent a distinct chemical class of natural products that contain fifteen-carbon terpenoid frameworks combined with lactone functional groups. The sesquiterpene lactone structure may provide a rigid molecular scaffold that positions reactive functional groups in specific three-dimensional orientations that may enable selective interactions with enzyme active sites.

The exocyclic enone moieties present in sesquiterpene lactones may serve as electrophilic centers that undergo Michael addition reactions with nucleophilic amino acid residues in enzyme active sites. The exocyclic enone functionality may comprise an α,β-unsaturated carbonyl system where the double bond extends outside of the lactone ring structure, creating an electrophilic carbon center that may be attacked by nucleophilic sulfur, nitrogen, or oxygen atoms present in amino acid sidechains. The positioning of the exocyclic enone relative to the sesquiterpene framework may determine the selectivity and reactivity of the mechanism-based inhibition process.

Referring to FIG. 2, sesquiterpene lactone inhibitors identified through the screening method may include dehydrocostus lactone and parthenolide, both of which contain the characteristic exocyclic enone moieties that enable mechanism-based enzyme inactivation. The chemical structures shown may demonstrate the common structural features shared by sesquiterpene lactone inhibitors, including large ring systems fused to trans α-exo-methylene γ-butyrolactone groups. The structural similarities between these compounds may indicate that the sesquiterpene lactone framework provides a privileged scaffold for developing mechanism-based inhibitors against bacterial enzymes.

The sesquiterpene lactone chemical class may provide a defined framework for medicinal chemistry optimization approaches that may improve potency, selectivity, and pharmacological properties of mechanism-based inhibitors. The rigid terpenoid backbone may serve as a molecular template that positions functional groups in predictable spatial arrangements, enabling structure-activity relationship studies that systematically explore the effects of structural modifications on biological activity. The defined framework may facilitate rational drug design approaches that optimize inhibitor properties while maintaining the mechanism-based inhibition characteristics.

Medicinal chemistry optimization of sesquiterpene lactone inhibitors may involve modification of the exocyclic enone moiety to adjust electrophilicity and reactivity toward target amino acid residues. The electrophilic character of the enone system may be modulated through substitution patterns on the double bond or through incorporation of electron-withdrawing or electron-donating groups that influence the reactivity of the Michael acceptor. The optimization process may balance enhanced reactivity for improved potency against reduced selectivity that might result from increased non-specific reactivity.

The lactone ring system in sesquiterpene lactone inhibitors may provide additional opportunities for medicinal chemistry optimization through modification of ring size, substitution patterns, or stereochemical configurations. The lactone functionality may contribute to binding interactions with enzyme active sites through hydrogen bonding or electrostatic interactions that enhance binding affinity and selectivity. The ring system modifications may also influence pharmacological properties such as metabolic stability, solubility, and tissue distribution that affect therapeutic efficacy.

The sesquiterpene backbone may be modified through alterations in ring fusion patterns, stereochemical configurations, or functional group substitutions that may affect binding interactions with target enzymes. The terpenoid framework may provide multiple sites for chemical modification that may be explored systematically to optimize inhibitor properties. The structural diversity accessible through sesquiterpene modifications may enable development of inhibitor libraries that span a range of potency and selectivity profiles.

Selectivity optimization of sesquiterpene lactone inhibitors may involve incorporation of recognition elements that enhance binding specificity for target enzymes while reducing interactions with off-target proteins. The recognition elements may include hydrogen bonding groups, hydrophobic substituents, or charged moieties that complement specific binding sites in the target enzyme active site. The selectivity enhancements may reduce potential side effects and may improve the therapeutic index of mechanism-based inhibitors.

Pharmacological property optimization may involve modification of sesquiterpene lactone structures to improve absorption, distribution, metabolism, and excretion characteristics that influence therapeutic efficacy. The lipophilicity of sesquiterpene lactones may be adjusted through incorporation of polar functional groups or through modification of ring systems to achieve optimal balance between membrane permeability and aqueous solubility. The metabolic stability may be enhanced through strategic placement of substituents that block metabolically labile positions or through incorporation of bioisosteric replacements that resist enzymatic degradation.

The defined structural framework of sesquiterpene lactones may enable systematic exploration of structure-activity relationships through parallel synthesis approaches that generate focused libraries of related compounds. The common synthetic intermediates and reaction pathways used for sesquiterpene lactone synthesis may facilitate efficient preparation of analog compounds for biological evaluation. The systematic approach may accelerate identification of optimized inhibitors with improved therapeutic properties.

The mechanism-based inhibition characteristics of sesquiterpene lactones may be preserved during medicinal chemistry optimization through retention of the exocyclic enone functionality while modifying other structural elements to improve selectivity and pharmacological properties. The preservation of the reactive moiety may ensure that optimized compounds maintain the irreversible enzyme inactivation mechanism that provides sustained antimicrobial activity. The optimization process may focus on enhancing the complementary binding interactions while maintaining the covalent modification capability.

The sesquiterpene lactone chemical class may provide advantages for drug development through the availability of natural product starting materials and established synthetic methodologies for structural modification. Many sesquiterpene lactones may be isolated from plant sources or may be prepared through well-characterized synthetic routes that enable efficient access to analog compounds. The established chemistry may reduce development timelines and may provide cost-effective approaches for preparing optimized inhibitors.

The three-dimensional structure of sesquiterpene lactones may provide conformational rigidity that may enhance binding selectivity through precise positioning of functional groups relative to enzyme active site features. The rigid framework may reduce conformational entropy penalties associated with binding while providing predictable spatial relationships between recognition elements and reactive groups. The conformational constraints may enable design of inhibitors with enhanced selectivity profiles compared to more flexible molecular scaffolds.

The natural product origin of sesquiterpene lactones may provide evolutionary validation of the chemical scaffold for biological activity, suggesting that the structural framework has been optimized through natural selection processes for interactions with biological targets. The natural product precedent may indicate that sesquiterpene lactones possess inherent biocompatibility and may exhibit reduced toxicity compared to purely synthetic chemical scaffolds. The evolutionary optimization may provide confidence in the therapeutic potential of the chemical class.

The sesquiterpene lactone framework may enable development of mechanism-based inhibitors with improved resistance profiles compared to reversible inhibitors, as the covalent modification mechanism may be less susceptible to resistance mutations that affect binding affinity. The irreversible nature of the enzyme inactivation may require more substantial genetic changes for resistance development, potentially extending the useful therapeutic lifetime of sesquiterpene lactone-based antimicrobial agents. The resistance advantages may provide strategic benefits for addressing antibiotic resistance challenges in bacterial pathogens.

### EXAMPLES

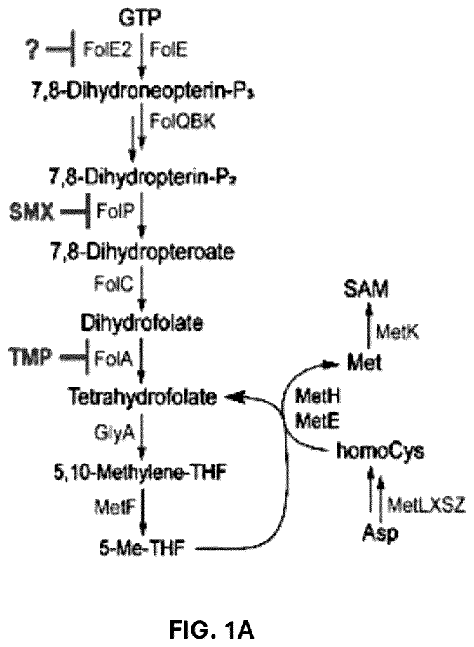

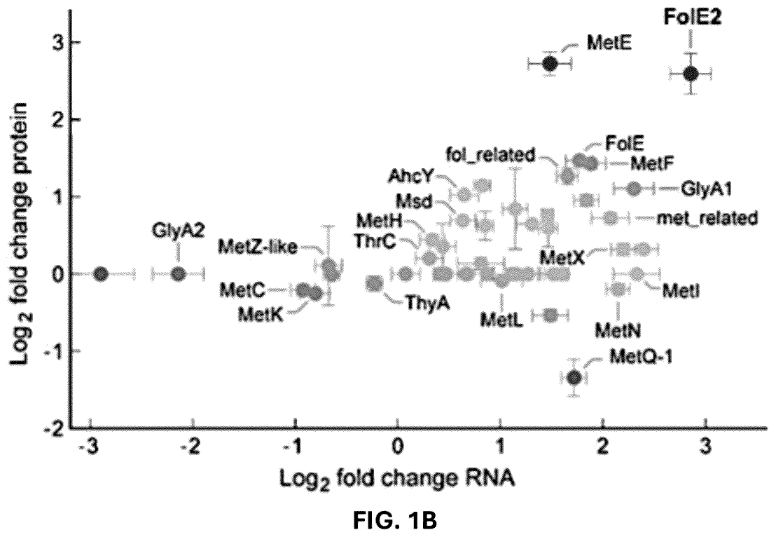

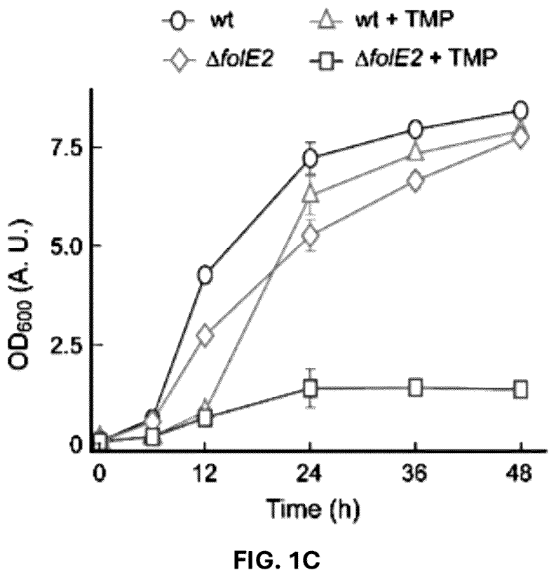

FolE2 is required for TMP tolerance. Previous studies revealed that low-dose TMP, much like inhibitory doses, exerts its effects through one-carbon metabolism, specifically by inhibiting dihydrofolate reductase (DHFR, FIG. 1A). Inspection of the inventors' prior transcriptomic and proteomic data show that folE2 is among the most upregulated genes in this pathway (FIG. 1). FolE2 and a related gene folE encode an inducible, Zn2+-independent and housekeeping, Zn2+-dependent GTP cyclohydrolases, respectively, which catalyze the first and rate-determining step in the biosynthesis of the essential cofactor tetrahydrofolate (THF). Under normal growth conditions, only FolE is required for THF production. Because TMP serves as an antimetabolite that interferes with the final step of folate biosynthesis, folE2 upregulation may represent a rescue mechanism for B. thailandensis to maintain growth in folate-depleted conditions.

To test FolE2's role in the presence of TMP, the inventors created a folE2 deletion mutant in B. thailandensis (ΔfolE2) and compared its growth to that of the parental wild-type (wt) in the presence and absence of subinhibitory levels of TMP. Neither the ΔfolE2 mutant nor treatment of wt B. thailandensis with low-dose TMP significantly affected growth. However, the combination of ΔfolE2 with subinhibitory concentrations of TMP resulted in a severely growth-inhibited phenotype. Ectopic expression of folE2 in the ΔfolE2 strain rescues this synthetic lethal phenotype (FIGS. 1C, 8A, 8B). Thymine and thymidine, however, fail to do so, consistent with previous reports. These results indicate that B. thailandensis either lacks sufficient uptake to rely on exogenous thymine or thymidine or does not undergo thymineless death under folate stress. The necessity of FolE2 in the presence of low-dose TMP suggests that the enzyme is an antimicrobial target; it has to date not been exploited for that purpose clinically. As noted above, adding to FolE2's appeal as an antimicrobial target is its absence from eukaryotic genomes. FolE homologs, however, are present in eukaryotes where they help generate biopterin, a cofactor involved in neurotransmitter biosynthesis. Loss of human FolE causes the dopamine-responsive dystonia referred to as Segawa syndrome.

Identification of possible FolE2 inhibitors. While folE is ubiquitous, folE2 is present in only ˜20% of microbial genomes. It is notably absent from many commensal gut bacteria, suggesting that FolE2-encoding pathogens may be targeted with some degree of specificity without affecting normal gut flora. The inventors imagined that a combination therapy using a FolE2 inhibitor along with subinhibitory dose of TMP could provide an avenue to selectively target Pseudomallei group pathogens, which include Burkholderia pseudomallei, B. mallei, and the non-pathogenic model B. thailandensis. The subinhibitory concentration of TMP would not adversely affect the growth of other bacteria, while increasing Burkholderia's reliance on FolE2. Inhibition of FolE2 would then cause a folate-less death only in TMP-susceptible bacteria that rely on FolE2 under folate-limiting conditions.

To test this idea, the inventors set out to identify FolE2 inhibitors in B. thailandensis. Identification of an inhibitor can be conducted directly in vitro using enzymatic assays or in vivo by measuring growth in the presence and absence of TMP. The inventors favored the latter approach as it gives a direct measurement of the desired endpoint, growth inhibition, and allows us to avoid issues of translating in vitro hits into in vivo leads. This was an especially important consideration given the challenges associated with small molecule penetration through the cell envelopes of Gram-negative bacteria.

The inventors screened a collection of ˜1,300 structurally diverse natural products and FDA-approved small-molecule drugs, a combination of two commercially available and routinely used libraries in high-throughput screens (see Methods, below), with the goal of identifying compounds that recapitulate the synthetic lethal phenotype of ΔfolE2 and TMP. The screen was carried out separately in the presence or absence of subinhibitory doses of TMP in biological duplicates; growth was assessed using optical density at 600 nm (OD600) after defined periods. Each experiment contained a negative control (DMSO vehicle) as well as a positive control (ΔfolE2). The average OD600 was then plotted in the presence and absence of TMP and growth-defects were defined as those with an OD600 that was two standard deviations below that of the vehicle control. The integrated results show that some compounds indeed recapitulated the ΔfolE2 plus TMP phenotype. Several compounds, likely antibiotics, exhibited growth inhibition in the presence and absence of TMP; these were disregarded. More importantly, 26 compounds exhibited no growth inhibition in the absence of TMP, but two or more standard deviations reduction in growth in the presence of subinhibitory TMP (FIG. 2). These leads were selected for further analysis.

Validation of inhibitors. To validate the results of the primary screen, the inventors re-assayed the hits in triplicate in both 96-well plates and flask cultures, as Burkholderia growth (and bacterial growth in general) is more reproducible in the latter. Unfortunately, five hits (triazolam, micafungin, mitomycin C, clonazepam, and gloriosine, were not commercially available in quantities to support validation cultures; the inventors therefore focused on the remaining 21 compounds (see, e.g., FIG. 9). The secondary screen validated ten compounds (FIG. 2), which are not toxic individually but lethal in combination, in both growth formats. For the other eleven compounds, the inventors recapitulated the results in one or the other growth format, but not both. The ten lead compounds compose a structurally diverse set of both natural products and FDA-approved drugs (FIG. 2). Promisingly, several structurally homologous compounds can be identified. For example, the natural products dehydrocostus lactone (DHL) and parthenolide are both sesquiterpene lactones, which possess large rings fused to trans α-exo-methylene γ-butyrolactones. These sesquiterpenes have been captured as hits in other screens as well, owing to the reactivity of the exocyclic enone. DHL is known for its antiproliferative properties against several cancer cell lines. It exhibits polypharmacology as it can induce both mitochondrial and endoplasmic reticulum stress-mediated apoptosis. Its activity is significantly reduced against non-cancerous cells. Miconazole and butoconazole, canonical inhibitors of ergosterol biosynthesis via lanosterol 14α-demethylase, are both clinically used imidazole-class azole antifungal agents. Finally, β-lapachone, osthole, and mefloquine all contain fused bicyclic, aromatic ring systems; the latter is used as an antimalarial agent. While some of these compounds have known antimicrobial activity, none are clinically used as antibacterial agents, nor do the inventors observe any antibacterial activity against B. thailandensis in the absence of TMP. For example, at 20 μM DHL or 20 μM TMP, no growth inhibition was observed. However, the combination of the two drugs resulted in >90% growth suppression (FIGS. 3A, 3B), a phenomenon referred to as chemical synthetic lethality to differentiate it from genetic synthetic lethality, which refers to the inactivation of two genes that are lethal only when combined. Note that (chemical) synthetic lethality is an umbrella term that applies to severe bacteriostatic growth inhibition as well, as the inventors observe in FIG. 3A.

To establish a causal relationship between administration of each of the ten compounds and growth inhibition, the inventors carried out dose-response analysis with varying concentrations of both TMP and the putative inhibitors. Sigmoidal, dose-dependent growth inhibition is observed only in the presence of TMP. Measurements of half-maximal inhibitory concentrations (IC₅₀) gave values between 1.7-11.8 μM in the presence of 20 μM TMP across the ten validated hits (FIG. 7). For each hit compound, the inventors also monitored IC₅₀ as a function of TMP concentration and found it generally to be inversely correlated with the TMP-dose. That is, increasing the dose of TMP greatly reduces IC₅₀ and thereby increases the potency of the inhibitors. With DHL, for example, the inventors observed a >100-fold reduction in IC50, from 271 μM to 1.8 μM, when TMP concentrations were raised from 5 μM to 20 μM; this range of TMP is subinhibitory in isolation (FIGS. 3C, 3D). The lower IC50 likely reflects the enhanced reliance on FolE2 for growth with increasing TMP doses.

Importantly, the inventors' compounds were completely non-inhibitory in the absence of TMP, even at concentrations several orders of magnitude higher than their respective IC50s in the presence of TMP. The inventors grew resistant mutants to the combination of TMP/DHL by subculturing B. thailandensis over several rounds. The mutants that emerged showed enhanced resistance to TMP. While the inventors' combinations require sensitivity to TMP, preexisting resistance to TMP is exceptionally rare with less than 1% of clinical isolates demonstrating resistance. Because the compounds do not affect growth without TMP, and therefore do not exhibit a minimal inhibitory concentration, the inventors cannot calculate a fractional inhibitory concentration index, making this co-treatment distinctly different from the synergistic combinations often used. Chemical synthetic lethality is also distinct from antibiotic adjuvant therapy, which seeks to block resistance pathways to a specific antibiotic with a second inhibitor (25, 26). The reliance upon TMP for activity corroborates the inventors' theory that FolE2 is part of a rescue mechanism under conditions of folate depletion.

Mechanism of FolE2 inhibition. The inventors tested whether the inhibitors act directly via inhibition of FolE2 using in vitro enzymatic assays. B. thailandensis folE2 was cloned, recombinantly expressed in E. coli, and purified by metal affinity and size-exclusion chromatography. With pure enzyme in hand, the inventors conducted enzymatic assays by monitoring formation of the FolE2 product, dihydroneopterin triphosphate, spectrophotometrically using its unique UV-visible absorption feature at 330 nm (ε=6,300 μM−1 cm−1). Steady-state kinetic analysis of FolE2 revealed apparent Km and kcat values of 22 μM and 74 min−1 (FIG. 4A), respectively, which are comparable to the values reported for FolE2 from B. subtilis. Having reconstituted the activity of FolE2, the inventors conducted enzymatic inhibition assays with each of the ten compounds. The inventors found three compounds—β-lapachone and the structurally analogous sesquiterpene lactones, DHL and parthenolide—inhibit the enzyme in vitro. Double reciprocal plots were then generated to characterize the inhibition mechanism of each compound, with β-lapachone acting as a noncompetitive inhibitor with respect to GTP, suggesting binding away from the active site, while both sesquiterpene lactones were uncompetitive with respect to substrate with apparent inhibition constants (Ki) ranging from 3-87 μM (FIGS. 4A-4E). Mechanism-based inhibitors manifest as uncompetitive inhibitors and the inventors hypothesized this inhibition mechanism may occur as a result of covalent modification of the enzyme through the common exocyclic enone found in both compounds. These additional hits indicate that TMP can form additional chemical synthetic lethal combinations with compounds that act upon different targets. Of special interest are miconazole and butoconazole, which exhibit low IC₅₀s in the presence of TMP (FIG. 7).

Crystal structures of DHL-inhibited FolE2. To explore the possibility of a covalently inhibited FolE2 with DHL, the inventors conducted ESI-MS and MALDI-ToF-MS experiments before and after treatment of the enzyme with the inhibitors. However, the inventors were not able to observe any modifications, suggesting that these may be lost due to in-source fragmentation or that DHL is not a covalent inhibitor. Interestingly, the active site Cys residue (Cys154 in Neisseria gonorrhoeae and Cys156 in B. thailandensis) has been reported to be nitrosylated in the N gonorrhoeae enzyme; this modification was also not observed by MS, which again may occur due to in-source fragmentation. We, therefore, explored inhibition by DHL further using X-ray crystallographic analysis. As FolE2 from B. thailandensis proved recalcitrant to crystallization, these experiments targeted the ortholog from the human pathogen B. pseudomallei, which is >94% identical to the B. thailandensis enzyme. Using vapor diffusion, the inventors successfully crystallized two different metalation states of FolE2, containing Na+ in the as-purified enzyme or Mn2+ upon incubation with the transition metal, as well as the DHL-inhibited form; their structures were subsequently solved to 2.02 Å, 2.82 Å and 3.10 Å, respectively.

The resultant models reveal a homotetrameric complex similar to that of the only other structurally characterized FolE2 from N. gonorrhoeae, in which two 16-stranded antiparallel β-barrels are arranged end-to-end to form an extended tunnel (FIG. 5A). Each barrel can be further broken down to the monomeric form of the enzyme, comprised of a bimodular T-fold core that is supplemented by two additional α-helices and one additional β-strand. Positioning of active sites at each three-subunit interface is also retained, as is the mode of metal ion coordination by two residues from one monomer (Cys154 and His166) and one (Glu208) from the adjacent β-barrel in the two apo structures (i.e. without DHL, FIG. 5B). Unlike previous studies, however, lack of additional coordinating ligands places the metallocofactor in a distorted trigonal pyramidal geometry. Identification of the bound metal ions was accomplished via careful inspection of the coordination environment and evaluation of inductively coupled plasma mass spectrometry (ICP-MS) data.

Just downstream of the metal-coordinating cysteine, the inventors observe electron density consistent with posttranslational nitrosylation of Cys156 (FIG. 5B). Despite inclusion of reductant in the crystallization buffer, this feature is present in all models, excluding only subunits for which the electron density of the sidechain is poor. Site-specific S-nitrosylation of the same conserved cysteine was previously reported in N. gonorrhoeae FolE2, and the inventors therefore assign the excess density as an S-nitroso group (24). While the physiological significance of this modification remains unclear, Paranagama et al. demonstrated a slight decrease in activity upon denitrosylation and proposed that the radical thionitroxide form of Cys156, stabilized by a nearby Arg, serves as a putative active site base. Arg205 may provide similar stabilizing interactions in the B. pseudomallei ortholog, however, its guanidino group is disordered, complicating interpretation. Furthermore, the inventors' models of the metalated enzyme depict the S-nitroso group in an approximately planar syn-conformation (CP-Sy-N-O dihedral angles between −8 and −34°), more consistent with an oxidized S-nitrothiol. Whatever its role in catalysis, S-nitrosylation at the same position in FolE2 orthologs suggests broader relevance of the modification.

Surprisingly, the inventors observe a second oxidative posttranslational modification in the structure of the as-purified, apo enzyme. Positive difference density on either side of the sulfur atom from Cys154 led to its subsequent identification as a sulfinate group (FIG. 5B). Although quite unusual, there is growing evidence that hyperoxidative modifications can be physiologically relevant. Both nitrile hydratases and thiocyanate hydrolase, for example, utilize a sulfinylated cysteine for metal coordination, and it has been hypothesized that changes in cysteine redox state may modulate the enzymes' affinities for different metals. It is not clear whether sulfinylation of Cys154 is merely an artifact of purification in FolE2 or whether oxidation of the metal-coordinating ligand plays a functional role. No other significant differences are observed between the Na+ and Mn2+ forms of the FolE2, nor with N. gonorrhoeae FolE2 (chain A of PDB accession code 3D2O). Alignment of the models yields root-mean-square deviations (rmsds) of the peptide backbone <0.45 Å over 245 Cα atoms. While large-scale structural features of the DHL-bound enzyme are similarly invariant (rmsds ˜0.60 Å), interaction with the inhibitor significantly disrupts the active site, leading to displacement of the metal ion (Mn2+ in this case) and increased structural disorder. Excitingly, the inventors observe binding of DHL and covalent modification of the enzyme in three of the inventors' subunits of the homotetramer. The degree to which this occurs varies by chain, perhaps due to differences in solvent access. In all modified molecules, the electron density is best fit by reduced Cys154 covalently crosslinked to DHL at the exo-enone, suggesting that this moiety undergoes Michael addition by the catalytic Cys154 to generate the inhibited enzyme (FIGS. 5C, 5D). Buffer exchanging the inhibited FolE2 led to nearly complete recovery of enzymatic activity, consistent with the conclusion above and the reversible nature of Michael addition reactions.

The resultant orientation of DHL further places the γ-butyrolactone group within close proximity to the metal-coordinating His166. Reaction of the imidazole sidechain with the γ-butyrolactone group could in theory generate an additional crosslink via the formation of a stable amide bond. However, electron density between DHL and the imidazole sidechain remains inconclusive at this resolution. Current models are instead more supportive of hydrogen-bonding interactions with both lactone-oxygens. Electron density for chains C and D, by contrast, appears to suggest the possibility of a second crosslink with another conserved histidine (His253), rationalized perhaps by oxidation of DHL to generate another conjugated system that can undergo Michael addition. Such a modification could be facilitated by Glu250, proposed to serve a similar role as an active site base during catalysis. The above interactions likely form the basis for DHL's remarkable specificity, as they occur between the inhibitor and the inventors' highly conserved catalytic residues. Thus, the inventors were somewhat surprised to find that DHL does not occupy the same binding site as the native substrate GTP. Rather, it extends vertically from Cys154 into an open pocket above the plane of the purine ring system that has been previously targeted for the rational design of improved inhibitors with greater specificity to FolE2 as compared to FolE. This arrangement is more reminiscent of that of TRIS depicted in previous structures of N. gonorrhoeae FolE2 (PDB accession code 5K9G) and hypothesized to mimic aspects of a putative reaction intermediate. A sulfite ion occupies a similar position in chain A of the inventors' structure, wherein Mn2+ has not been displaced by DHL. The inventors hypothesize that excess difference density observed to overlay with DHL in all other chains corresponds to a mixture of DHL-modified and Mn2+/sulfite-bound enzyme, but precise occupancies could not be determined. Thus, the minor unmodified component was left unmodeled.

Together, the results above demonstrate that β-lapachone, DHL and parthenolide form a chemical synthetic lethal combination with low-dose TMP by inhibiting FolE2. As supported by crystallographic and enzymatic inhibition studies, DHL is a covalent inhibitor that undergoes Michael addition at its electrophilic exo-enone moiety to inactivate FolE2.

Chemical synthetic lethality in B. pseudomallei. After establishing that three compounds—DHL, parthenolide, and β-lapachone—exhibit a chemical synthetic lethal combination by targeting FolE2 in B. thailandensis, the inventors moved to recapitulate these results in the human pathogen B. pseudomallei JW270. Excitingly, the inventors found that DHL and β-lapachone exhibit a similar effect. In both cases, no growth inhibition was observed when B. pseudomallei was treated with low-dose TMP, DHL or β-lapachone alone. In combination, however, the inventors observed a significant drop in IC₅₀ to 2.5 and 3.2 μM with DHL or β-lapachone, respectively, in co-treatment with low-dose TMP (FIG. 7). The comparable activities observed for these FolE2 inhibitors in B. thailandensis and B. pseudomallei suggest that the metabolic response to sublethal doses of TMP is conserved. By contrast, parthenolide/TMP, which showed activity similar to DHL/TMP in B. thailandensis, did not inhibit B. pseudomallei. The folE2 sequences across the two strains is 94.6% and 97.8% identical at the DNA and protein levels, respectively, and no differences are present in the active site sequence. Therefore, it seems likely that the loss of activity with parthenolide/TMP is due to differences in cell envelope permeability rather than differences between the two folE2 orthologs. The different ring sizes of DHL and parthenolide, as well as the 30 kb capsule operon deletion in the B. pseudomallei strain that the inventors used, further support this hypothesis.

As a final test of the new combinations, the inventors examined the susceptibility of select commensal bacteria in the gut. Of the strains tested, TMP/DHL inhibited the growth of E. coli only (FIG. 6). Significantly, the first-line combination therapy of TMP/SMX was more growth-inhibitory than TMP/DHL across all strains tested, with the exception of E. coli and E. faecalis. TMP/DHL was likewise found to be substantially more specific than secondary treatment options used for melioidosis, consisting of doxycycline, meropenem, and ceftazidime, which killed the majority of commensal strains tested. These results highlight the specificity of chemical synthetic lethal combinations and promote DHL/TMP and β-lapachone/TMP as leads for additional investigation and as useful small molecule tools to further study FolE2 as a conditionally essential antimicrobial target in B. thailandensis.

### DISCUSSION

In contrast to antiviral or anticancer therapies, where dual or triple combinations are common, clinical antibiotic treatment has been dominated by monotherapies. This approach has proven successful with Gram-positive pathogens and Gram-negative pathogens with small genomes. However, treatment of pathogens with large genomes, such as P. aeruginosa or B. pseudomallei, is much more challenging because resistance can emerge more rapidly in these organisms, a phenomenon attributed to the greater capacity for metabolic contingencies that comes with larger genomes. Herein, the inventors introduce a method for selecting combinations of molecules that are lethal in combination, but innocuous in isolation. These are selected not based on synergistic interactions or adjuvant therapy that targets resistance mechanism, but rather on chemical synthetic lethality.

Synthetic lethality, a lethal condition of two gene deletions only in combination, has been well studied. The inventors apply the chemical version of this phenomenon in B. thailandensis and B. pseudomallei by identifying binary small molecule combinations, TMP/DHL and TMP/β-lapachone, which halt the growth of both bacteria by inhibiting FolE2, an enzyme that becomes essential in the presence of subinhibitory levels of TMP. In addition to these FolE2 inhibitors, the inventors have discovered seven other compounds which recapitulate the synthetic lethal phenotype but do not affect FolE2; these compounds pose an attractive starting point for discovering previously unknown targets for antibiotic action. While FolE2 and DHFR are both components of the folate pathway, TMP's pleiotropic activation of Burkholderia secondary metabolism by partial inhibition of primary metabolism may allow us to target a multitude of pathways that are not obviously related to folate biogenesis.

A caveat of the work is that subinhibitory concentrations of TMP were used to change the metabolic requirements of Burkholderia. While TMP is not toxic at the subinhibitory doses used, it nonetheless has an impact on the bacteria, even if this impact is not read out by OD₆₀₀ measurements. This mirrors the genetic equivalent of the method, in which a given mutation alters metabolism, but not growth, to make the second deletion lethal. It remains to be seen whether (chemical) synthetic lethality without this qualifying condition is possible. The advantage of the method as presented is that much lower TMP concentrations are used. This approach leaves many bacteria, especially commensal or beneficial ones, unscathed, while Burkholderia are susceptible toward DHL treatment. The appeal of this combination is seen in screens of commensal bacteria. Although much more susceptible to antibiotics than Gram-negative pathogens, Burkholderia are effectively inhibited by TMP/DHL, but this combination does not affect the growth of most commensal strains tested, such as B. fragilis and B. longum (FIG. 6).

Similar synthetic lethal combinations have previously been identified, largely in methicillin-resistant Staphylococcus aureus (MRSA). Specifically, oxacillin and tunicamycin were found to exhibit this type of phenotype via inhibition of cell wall transpeptidase PBP2 and cell wall teichoic acid glycosyltransferase TarO. Similarly, cefuroxime and ticlopidine were identified as a synthetic lethal combination in MRSA, with the molecules also targeting PBP2 and TarO, respectively. Other combinations in S. aureus have been described as well, as have sensitization screens in E. coli to render the bacterium susceptible to Gram-positive antibiotics using adjuvants or to leverage synergistic interactions with reactive oxygen species-generating drug molecules. The inventors' study is different in that it relies on a conditionally essential gene that is not widely distributed and concentrations of compounds which do not affect other microorganisms, greatly increasing specificity. Moreover, it targets Gram-negative bacteria with large genomes (>6.5 Mbp) that are difficult to treat with conventional antibiotics.

Chemical synthetic lethality allows us to expand the repertoire of antibiotics to targets that are otherwise non-druggable. While traditional therapies target pathways that are widely distributed among bacteria, the inventors' strategy relies on perturbing bacterial metabolism to create unique or uncommon secondary targets that are otherwise nonessential. By relying on the metabolic idiosyncrasies of a pathogen of interest, the inventors can narrow the spectrum to only those bacteria which have an identical metabolic response. In doing so, chemical synthetic lethality reduces off-target effects, making it significantly more specific than traditional antibiotic therapy.

### Materials and Strains

Bacterial Strains and Culture Conditions. Burkholderia thailandensis E264, acquired from the American Type Culture Collection (ATCC 700388), was the primary bacterial strain used in this study, unless noted otherwise. Initial culture involved streaking the strain onto an 1.5% LB agar plate (Fisher Scientific) and incubating it overnight at 37° C. Subsequently, 5 mL of LB-MOPS medium—consisting of LB (Becton Dickinson) buffered to pH 7.0 with 50 mM MOPS and NaOH (Fisher Scientific)—in a 14 mL culture tube was inoculated from the agar plate. These tubes were then incubated at 30° C. with continuous shaking at 200 rpm and a stroke length of 25.4 mm, again for an overnight period.

Escherichia coli Strains for Plasmid Construction and Expression. For the purposes of plasmid construction, amplification, and maintenance, Escherichia coli DH5α was utilized; E. coli BL21 was employed for heterologous expression. Both strains were cultured at 37° C./200 rpm.

Selective growth was facilitated using tetracycline (20 μg/mL), steptomycin (100 μg/mL), and kanamycin (50 μg/mL).

Commensal Bacterial Strains. Cultivation of these strains was conducted anaerobically using a GasPak pouch-based system (Becton Dickinson) at 37° C. in pre-degassed Nissui Modified Gifu Anaerobic Medium broth (HyServe).

Cloning. For genetic manipulations, restriction enzymes, proofreading Q5 DNA polymerase, HiFi DNA Assembly Master Mix, calf intestinal alkaline phosphatase (CIP), and associated buffers were procured from New England Biolabs. PCR reactions were routinely performed using Failsafe Buffer G (Epicentre). A comprehensive array of reagents including primers, antibiotics, IPTG, GTP, MnCl2, imidazole, β-mercaptoethanol (OME), phenylmethylsulfonyl fluoride (PMSF), and a protease inhibitor cocktail were sourced from MilliporeSigma. Additional chemicals such as HEPES, Tris, dithiothreitol (DTT), glycerol, and KCl were obtained from Fisher Scientific.

Spectrophotometric, HPLC-MS, and Sequencing Analysis. UV-visible absorption spectra were recorded using a Cary 60 UV-visible spectrophotometer (Agilent) with standardized 1 cm pathlength cuvettes. For plate-based assays, a Synergy H1 Hybrid Multi-Mode Microplate Reader (Biotek) was employed. Low resolution high-performance liquid chromatography-mass spectrometry (HPLC-MS) analysis was performed on an Agilent instrument consisting of a liquid autosampler, a 1260 Infinity Series HPLC system coupled to a diode array detector, and a 6120 Series ESI mass spectrometer. A Phenomenex Luna C18 column (3 m, 4.6 mm×100 mm) was used with a flow rate of 0.5 mL/min and the following elution plan: 10% MeCN in water for 3 min, 10-100% MeCN over 20 min. H2O and MeCN contained 0.1% (v/v) formic acid (FA). Sequencing services were provided by Genewiz.

Generation of B. thailandensis ΔfolE2 mutant. The ΔfolE2 mutant was generated employing natural competence transformation. This entailed transforming B. thailandensis with a linear DNA fragment, incorporating an antibiotic resistance marker flanked by 1 kb homologous regions upstream and downstream of the folE2 gene. The procedure was adapted from established methods. Briefly, a linear fragment was generated by amplifying 1 kb regions upstream and downstream of folE2 using primers upstream.F and upstream.R for the upstream fragment and downstream.F and downstream.R for the downstream fragment and TetR.F TetR.R for the Tet resistance marker which were subsequently joined using overlap extension PCR using the same primers. The fused fragment was then purified and used to transform B. thailandensis. To do so, an overnight culture was used to inoculate 3 mL of M63 in a 14 mL culture tube which was grown at 37° C. 200 rpm. After 10 hours, the cells were centrifuged at 12 000×g for 2 min and resuspended in 100 μL of M63. About 500 ng of a linear DNA fragment was added to 50 μL of this suspension, followed by a 30-min room temperature incubation. The mixture was then cultured overnight in M63 medium at 37° C./200 rpm. Post-growth, the culture was centrifuged, and the pellet resuspended in 80 μL of M63 and plated on low-salt LB agar with tetracycline S2(20 g/mL). Mutant colonies were identified and confirmed through PCR and DNA sequencing after a 24 h incubation at 37° C.

96-well plate-based Burkholderia assays. Assays were routinely conducted in duplicate using either Fisher Scientific model 12-565-501 or Corning 3631 polystyrene 96-well plates. Prior to use, these plates underwent sterilization via UV irradiation. Overnight B. thailandensis cultures were prepared as described above and used to inoculate 25 mL of LB-MOPS in a 125 mL Erlenmeyer flask to an initial OD600 of 0.05. When necessary, TMP was added to this suspension from a 100 mM stock prepared in DMSO for a final concentration of 30 μM. This suspension was used to accurately fill 96-well plates to a final volume of 110 μL/well using a MultiFlo peristaltic reagent dispenser (BioTek). To mitigate risks of contamination and reduce evaporation, the plates were sealed with Breathe-EZ sealing membranes (MilliporeSigma). The plates were cultured at 37° C./200 rpm for ˜12 h and growth was subsequently quantified by measurement of OD600.

High throughput screen. The high-throughput screening approach involved the use of two distinct small molecule libraries: a natural product library consisting of ˜500 molecules (catalog no. BML-2865, Enzo Scientific) and an FDA-approved drug library consisting of ˜800 molecules (catalog no. BML-2843, Enzo Scientific). These libraries encompass a wide range of structurally and functionally diverse compounds, including natural products and FDA-approved small molecules. To conduct the screen, 0.4 μL of each compound from these libraries was dispensed into duplicate 96-well plates containing B. thailandensis (or B. thailandensis plus TMP), prepared as described above, using the Cybi well HTS robotic multichannel pipettor system (CyBio), resulting in final concentrations of 7.3 μg/mL for the natural products and 36 μM for the FDA-approved drug compounds. The plates were sealed with Breathe-EZ sealing membranes and incubated at 37° C./200 rpm. Cell density was closely monitored at time intervals of 0, 6, 12, 24, and 48 h post-treatment. Special emphasis was placed on the 24 h and 48 h measurements to assess growth inhibition effectively. In these assays, control groups included both untreated wt and the ΔfolE2 mutant, providing essential baselines for comparison.

Validation of hits & IC50 assays. Plate-based validations closely replicated the conditions established in the initial high-throughput screen, with the exception that they were set up in triplicates. Flask validations were conducted under upscaled standardized conditions: A 5 mL overnight B. thailandensis culture, prepared as described above, was used to inoculate 25 mL of LB-MOPS in 125 Erlenmeyer flasks to an initial OD600 of 0.05. Compounds of interest were added from DMSO stocks; final concentrations are indicated in the figures. Vehicle-control flasks (carrying the same volume of DMSO) were included in triplicates as well. These were then cultured 37° C./200 rpm for 12-24 h, depending on the experiment, and OD600 was subsequently determined. The IC50 values for each compound were measured in 96-well plates by creating half-log serial dilutions of the compounds, beginning at a concentration of 100 μM, prepared from a 100 mM DMSO stock. For TMP, 2:1 serial dilutions were performed, starting at 20 μM. The final concentration of DMSO in these assays was maintained at 0.2%. The IC50 values were calculated using the formula:

$$\frac{{growth}_{max}}{1 + \frac{[ compound ]}{{IC}_{50}}} x .$$

The calculation was executed using the NonlinearModelFit function in Mathematica (Wolfram Research), and the modelled dose-dependence curves were plotted using Mathematica.

Complementation of folE2. The folE2 gene (locus tag: BTH_II0615) was PCR-amplified from the genomic DNA of B. thailandensis E264 using Q5 High-Fidelity DNA Polymerase, and primers folE2.F and folE2.R, and purified using a PCR purification kit (Qiagen). The pDMrhaB2 expression vector was linearized by PCR using primers pDMrhaB2.FOR and pDMrhaB2.REV and purified by gel extraction kit (Qiagen). The ligation was facilitated using HiFi Assembly, resulting in the construct pDMrhaB2-folE2 which was transformed in E. coli DH5α. Following transformation, selection was conducted by incubating the transformed cells on LB agar plates supplemented with kanamycin overnight. A single colony from this selection was then cultured overnight in 5 mL of LB medium with kanamycin selection. The plasmid was extracted from this culture using the QIAprep Spin Miniprep Kit (Qiagen) and its integrity confirmed by rapid plasmid sequencing by Genewiz. The pDMrhaB2-folE2 and empty pDMrhaB2 plasmids were then transformed into E. coli JV36 cells, and conjugation with wild-type and ΔfolE2 mutant was conducted as previously described selecting with streptomycin and kanamycin. A single colony of each of the inventors' strains, wild-type/ΔfolE2 and expression/empty vector, was selected and was used to inoculate a 14 mL culture tube containing 5 mL LB medium supplemented with kanamycin overnight. These starter cultures were diluted and dispensed into 96-well plates with either 20 μM TMP or a DMSO vehicle control for a final starting OD600 of 0.05 with 0.2% rhamnose before being grown under standard conditions. Growth was evaluated after 48 hours using OD600 (see FIGS. 8A, 8B).

Supplementation of thymine and thymidine. Wild-type and ΔfolE2 mutant overnight starter cultures prepared as previously described were diluted and dispensed into 96-well plates for a final starting OD600 of 0.05. To the ΔfolE2 mutant was added either 20 μM TMP or a DMSO vehicle control, and to the wild-type was added either DMSO vehicle control, TMP/DHL, TMP/miconazole, or TMP/SMX at each combination's respective MIC. Finally, either thymine or thymidine was in half-log serial dilutions of the compounds, beginning at a concentration of 1 mM, prepared from a 100 mM DMSO or water stock respectively. Final DMSO concentrations were normalized across all conditions. Each condition was grown in quadruplicate under standard parameters and was evaluated after 48 hours using OD600 (see FIGS. 8A, 8B). A modification was also attempted where cultures were preincubated for 1 hour in thymine or thymidine before inhibitor was added but showed similar results and is therefore not included.

Cloning folE2.

B. thailandensis. The folE2 gene (locus tag: BTH_II0615) was PCR-amplified from the genomic DNA of B. thailandensis E264 using Q5 High-Fidelity DNA Polymerase, Failsafe Buffer G, and primers folE2.pET28b.SacI.R and folE2.pET28b.NdeI.F. The PCR products were then purified using a PCR purification kit (Qiagen) and subsequently ligated into the pET-28 b (+) expression vector, which had been linearized through double digestion with NdeI and SacI-HF for 1 h at 37° C. in CutSmart buffer. The ligation was facilitated using HiFi Assembly, resulting in the construct pET-28-b-(+)-6xHis-folE2. This construct was then transformed into E. coli DH5α. Following transformation, selection was conducted by incubating the transformed cells on LB agar plates supplemented with kanamycin overnight. A single colony from this selection was then cultured overnight in 5 mL of LB medium. The plasmid was extracted from this culture using the QIAprep Spin Miniprep Kit (Qiagen) and its integrity confirmed by Sanger sequencing using T7 primers by Genewiz. The plasmid was then transformed into E. coli BL21 (DE3) cells, and expression carried out as described below.

B. pseudomallei. A pET28a(+) plasmid containing an N-terminally 6x-His tagged folE2 gene from B. pseudomallei JW270 was obtained from Twist Biosciences. After verifying its sequence, the plasmid was used to transform E. coli BL21 (DE3), followed by selection on LB agar/kanamycin plates.

### Expression and Purification of FolE2.

B. thailandensis. A single colony of E. coli BL21 harboring vector pET-28-b-(+)-6xHis-folE2 was used to inoculate a 14 mL culture tube containing 5 mL LB medium supplemented with kanamycin. This culture was grown for 12 hours, after which a 500 μL aliquot (1% v/v) of the overnight culture was subcultured into 50 mL of LB medium/kanamycin in a 250 mL Erlenmeyer flask. The intermediate culture was allowed to grow for an additional 8 hours. For large-scale expression, the inventors' 2.8 L Fernbach flasks were prepared, each containing 750 mL of LB medium/kanamycin. Each flask was inoculated with 750 μL (0.1% v/v) of the intermediate culture. These were grown to an OD600 of 0.6. Subsequently, the cultures were cooled in an ice bath for 10 minutes before the addition of IPTG to a final concentration of 100 μM. Following induction, the flasks were returned to the incubator and cultured at 18° C./200 rpm. After 12 hours, the cells were harvested by centrifugation (8,000 g, 15 min, 4° C.). The cell pellets were stored at −80° C. The purification of FolE2 was conducted at 4° C. according to previously published protocols. The lysis buffer consisted of 50 mM Tris, 50 mM NaCl, 5 mM imidazole, 5% glycerol, pH 7.8, and 1 mM WE. The cell pellet was resuspended in lysis buffer (5 mL/g) in a 250 mL beaker, supplemented with 0.1% v/v protease inhibitor cocktail, 0.25 mM PMSF, 1 mg/mL lysozyme, and 10 U/mL DNase I. The suspension was stirred for 30 minutes and sonicated on ice in 15 s on/15 s off cycles at 30% power for a total of 2 min. This was followed by a 5 min rest period, and then a repeat of the sonication cycle. The cell debris was then pelleted by centrifugation (32,000 g, 1 h, 4° C.). PMSF was added to the supernatant at a final concentration of 0.25 mM; the mixture was then loaded onto a nickel metal affinity column (12 mL) pre-equilibrated with lysis buffer. The column was washed with 10 column volumes of lysis buffer and 4 column volumes of wash buffer (50 mM Tris, 50 mM NaCl, 30 mM imidazole, 5% glycerol, pH 7.8, 1 mM βME, and 0.25 mM PMSF). Elution of the protein was performed with 4 column volumes of elution buffer (50 mM Tris, 50 mM NaCl, 300 mM imidazole, 5% glycerol, pH 7.8, 1 mM WE, and 0.25 mM PMSF). FolE2 was buffer-exchanged on a 20 mL Sephadex G-25 column equilibrated with G-25 buffer (50 mM Tris, 100 mM KCl, 5% glycerol, pH 7.8). The desired protein fractions were identified by the Bradford assay (Bio-Rad), pooled, analyzed by SDS-PAGE (see FIGS. 3A-3D) and UV-vis spectroscopy, flash-frozen in liquid N2, and stored at −80° C.

B. pseudomallei. FolE2 from B. pseudomallei JW270 was expressed and purified in an analogous manner. A 1 L Erlenmeyer flask containing 500 mL of LB medium supplemented with 50 μg/mL kanamycin was inoculated with a single colony of E. coli BL21 (DE3) cells carrying pET28a(+)-6xHis-folE2. This culture was grown for ˜17 h at 37° C./230 rpm. Then, four 4 L Erlenmeyer flasks each containing 2 L of LB/kanamycin were inoculated with 25 mL of the starter culture per liter of LB and cultured at 37° C./230 rpm to an OD600 of 0.6. Protein expression was induced by adding 0.1 mM IPTG. The cultures were further grown at 16° C./230 rpm for 18-20 h. The cells were harvested via centrifugation (9,000 g, 20 min), flash-frozen in liquid nitrogen, and stored at −80° C. The frozen cell paste was thawed by resuspending in 5 mL of lysis buffer (20 mM HEPES, 400 mM NaCl, 10 mM βME, 10 mM imidazole, pH 7.5) per gram of cell paste. This solution was supplemented with 10 μg/mL of DNase I, protease inhibitor cocktail (1:10,000 dilution of stock), 1 mM PMSF, and 1 mg of lysozyme for every mL of solution. This mixture was stirred at 4° C. for at least 30 minutes until the mixture was homogenous. Cells were lysed via sonication (15 s on, 15 s off for 4 min of run time with 50% amplitude) at 4° C., stirred, then sonicated a second time under the same conditions. Cell debris was removed by centrifugation (32,000 g, 1 h, 4° C.), and the supernatant was directly loaded onto a HisPur Ni-NTA resin (Thermo Scientific) column containing 15 mL of packed resin pre-equilibrated with the lysis buffer. The column was washed with 10 CV of wash buffer (20 mM HEPES, 400 mM NaCl, 10 mM βME, pH 7.5) supplemented with 10 mM imidazole, followed by subsequent washes with 4 CV of wash buffer supplemented with 30, 50, 70, and 90 mM imidazole, respectively. The protein was eluted with 4 CV of 20 mM HEPES, 400 mM NaCl, 10 mM OME, 300 mM imidazole, pH 7.5. The eluate was concentrated using Amicon Ultra filters with a 10 kDa molecular weight cutoff. The protein was then buffer-exchanged into 20 mM HEPES, 400 mM NaCl, 10 mM ME, pH 7.5 using a PD-10 column (Cytiva). In order to obtain sufficient purity for crystallization, the enzyme was subsequently passed through a HiLoad 16/600 Superdex 200 pg size exclusion column (Cytiva) pre-equilibrated with 20 mM HEPES, 400 mM NaCl, 10 mM βME, pH 7.5 at 4° C. The protein was purified with an isocratic gradient of the buffer and elution monitored via absorbance at 280 nm. Fractions contributing to the highest absorbing peak were then analyzed via SDS-PAGE. The purest fractions were pooled together and concentrated to ˜20-22 mg/mL as confirmed by the absorbance at 280 nm using the molar extinction coefficient of 19,940 M⁻¹ cm⁻¹.

MS analysis and enzymatic activity assays. The mass of the purified enzyme was confirmed by buffer-exchanging into water using a Bio-Spin column (Bio-Rad). The mass analysis was then conducted using an ESI-TOF mass spectrometer (Agilent 6220). The measured mass of the enzyme was found to be 33207.16 Da, closely aligning with the expected mass of 33207.71 Da. To verify the enzyme's catalytic activity, 50 μM of enzyme was incubated with 1 mM GTP in reaction buffer (100 mM HEPES, 100 mM KCl, 0.5 mM MnCl2, and 1 mM DTT) at 37° C. for 1 hour. Post-incubation, the reaction mixture was treated with 5 units of calf intestinal alkaline phosphatase (NEB) and further incubated at 37° C. for 1 h. The resultant mixture, along with a standard of the authentic product, was analyzed by HPLC-ESI-MS using the system described above. The analysis revealed a major peak in the reaction mixture that matched the retention time and mass ([M+H]+=256.1) of the product standard, thus confirming the GTP cyclohydrolase activity of FolE2.

Michaelis-Menten kinetics. FolE2 was preincubated in clear-bottom black-sided 96-well plates (Corning 3631) at 37° C. using an assay buffer composed of 100 mM HEPES, 100 mM KCl, 0.5 mM MnCl2, and 1 mM DTT for 10 min. Subsequently, GTP was added, which was serially diluted, into each well in triplicates. This addition brought the final assay volume to 100 μL and set the enzyme concentration at 10 μM. The accumulation of the product, 7,8-dihydroneopterin triphosphate, characterized by its unique maximal absorbance (λₘₐₓ) at 330 nm (ε=6300 M−1 cm−1) (2), was measured to monitor the reaction progress. This measurement was performed using a kinetic spectroscopic experiment on a microplate reader. The rates of product accumulation were determined by linear regression and Vmax and KM were calculated using the formula: rate=Vₘₐₓ[GTP]/K_{M}+[GTP] using Mathematica's NonlinearModelFit function (Wolfram Research).

IC50 activity assays. Enzymatic inhibition assays were conducted in a similar format to the above-described assays with the only modification being the addition of either small-molecule inhibitor or vehicle (DMSO) during the preincubation of FolE2/GTP, the latter supplied at its K_{M}. The IC50s were calculated using the formula:

$$rate = \frac{{rate}_{max}}{1 + \frac{[ compound ]}{{IC}_{50}}}$$

using Mathematica's NonlinearModelFit function, and the modelled dose-dependence curves plotted using Mathematica.

Double reciprocal plots/Ki measurements. The Kᵢ was assayed using similar conditions as above with the modification being the systematic variation of substrate and inhibitor concentrations. The Ki was calculated based on the general equation

$$v = \frac{{V}_{max} [ S ]}{[ S ] + ( 1 + \frac{[ I ]}{α {K}_{i}} ) + {K}_{M}} .$$

Reversal of DHL inhibition. An enzymatic inhibition assay was performed following the previously established protocol with incubation of FolE2 with either vehicle (DMSO) or DHL at its IC₉₅ at 37° C. for 10 min. One set was immediately dispensed into 384-well plates with addition of GTP at its KM while another underwent size exclusion chromatography using Bio-Rad Mini Bio-Gel P-6 cartridges to remove unbound DHL followed by a 1 hr incubation in 384-well plates before GTP was added. FolE2 activity was calculated as previously described, and enzyme with DHL was normalized to its respective DMSO control.

B. pseudomallei antimicrobial assays. Experiments using Burkholderia pseudomallei JW270 were conducted using experimental conditions that were analogous to assays using B. thailandensis with the exception of growth at 240 rpm and the use of B. pseudomallei JW270, which has a mutation in the rpsL gene, a deletion of the amrAB-oprA efflux pump, and a deletion of the capsule gene cluster wcb for attenuation (BSL-2).

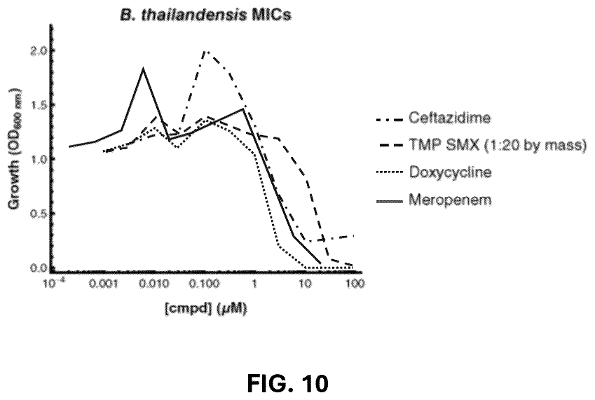

Commensal strains growth. The MICs of clinically used antibiotics against B. thailandensis were determined using standard methods (see FIGS. 6, 10). For the fixed dose combination of trimethoprim-sulfamethoxazole, a 1:20 ratio was used to most closely approximate the serum ratio. The commensal strains were directly inoculated into 14 mL culture tubes containing degassed modified GAM and incubated overnight at 37° C. in a GasPak anaerobic system. Simultaneously, a starter culture of B. thailandensis was grown under standard conditions.

The starter cultures were then dispensed into 96-well plates with either vehicle control, clinically used antibiotic, or synthetic lethal combination for a final culture OD600 of 0.05 and compound concentration at its MIC against B. thailandensis in sextuplicate. The synthetic lethal combinations were dosed at 20 μM of TMP and the MIC of the other compound. Anaerobic strains continued to be incubated at 37° in a GasPak anaerobic system while B. thailandensis was grown aerobically in standard conditions. Growth inhibition was assessed at 24 and 48 hours and normalized as fractional OD600 of the vehicle control.

Crystallization of FolE2 from B. pseudomallei. The as-purified enzyme was initially crystallized via hanging drop vapor diffusion at room temperature by mixing the enzyme (22 mg/mL) 1:1 with 0.1 μM HEPES sodium salt pH 7.5, 1.6 μM ammonium sulfate, and 2% PEG 1000 to generate a final drop volume of 2 μL. To improve crystal quality, a seed stock was subsequently produced by combining a single drop of the above crystals with 50 μL of reservoir solution and 50 L of FolE2 at 18 mg/mL, followed by brief vortexing in a Seed Bead Kit (Hampton). Seed stock dilutions up to 10-7 were made by mixing 0.2 uL of seed stock with a 1.8 uL drop containing a 0.8:1 ratio of protein/reservoir to yield a final volume of 2 uL. Crystals grew to full size and were harvested from the 10-3 dilutions drops within approximately 2 weeks. The resultant crystals were fairly small ˜50 microns in size with a hexagonal pyramidal structure. For structures with manganese bound, the protein was buffer-exchanged into 20 mM HEPES, 20 mM NaCl, 2.5 mM MnCl2, 2 mM βME, pH 7.5 using a PD-10 column (Cytiva) and concentrated to 20 mg/mL before setting up crystallization trays. Crystals were then grown via sitting drop vapor diffusion at room temperature. The protein was mixed in a 1:1 ratio with a precipitant solution of 0.1 M NaCl, 0.1 M Bis-Tris pH 6.3-6.8. 1.3-1.7 M ammonium sulfate. Five molar equivalents of dehydrocostus lactone (DHL) was also added to the protein stock solution to obtain crystals with the inhibitor bound. This enzyme-inhibitor cocktail was then mixed in a 1:1 ratio with the same precipitant solution containing 0.01 CdCl2 as an additive. In both cases, crystals typically formed within 1 week and reached full growth between 2-4 weeks after setup. The resultant crystals were harvested by looping and briefly transferred into a cryoprotectant comprised of their respective precipitant solutions containing an added 30% (v/v) of ethylene glycol before flash-freezing in liquid nitrogen. For crystals grown in the presence of DHL, 15 mM of DHL was included in the cryoprotectant.

X-ray data collection and processing. Diffraction data for the manganese and DHL-bound structures were collected at beamline 21-ID-G of the Advanced Photon Source (APS) at Argonne National Laboratory (Chicago, IL) using a Rayonix MX-300 CCD detector. Crystals were maintained at 100 K to minimize X-ray induced damage while images were collected sequentially (Δφ=1°) with an incident wavelength of 0.97857 Å. The data were indexed, integrated, scaled, and merged using HKL2000. Diffraction data for the sodium-bound structure were collected at beamline 23-ID-B of the APS with an Eiger X 16M (Dectris) detector. Crystals were again maintained at 100 K, however, these images were collected sequentially (Δφ=0.2°) with an incident wavelength of 1.033 Å. The data were subsequently indexed, integrated, and scaled using XDS before merging with AIMLESS. All structures were solved via molecular replacement with PHASER (5) using a previously solved structure of FolE2 from Neisseria gonorrhoeae FA 1090 (PDB accession code: 5K95) as the search model. Model-building was conducted in Coot,(7) and structures were refined in Phenix. Coordinates and restraints for DHL were generated in JLigand.(9) Model quality was assessed using MolProbity. Both structures solved in the absence of DHL were in the P3221 space group and contain two molecules in the asymmetric unit. The final model of the Mn-bound enzyme (PDB accession code: 86GC) was refined to 2.82 Å, while that of the Na+-bound enzyme (PDB accession code: 8G8V) was refined to 2.02 Å. The structure containing DHL was in the P212121 space group and contained four molecules in the asymmetric unit. The final model of the DHL-bound enzyme (PDB accession code: 8TCC) was refined to 3.10 Å.

ICP-MS Experiments and Metal Ion Identification in crystallo. To confirm metal identity in FolE2 crystals, ICP-MS was performed to test for Mn2+, Mg2+, Ni2+, and Zn2+. Toward this end, FolE2 was buffer exchanged into 20 mM HEPES, 400 mM NaCl, 10 mM BME, pH 7.5 using a PD-10 column (Cytiva). The enzyme was then denatured by heating at 95° C. for 10 minutes, and the aggregate removed via centrifugation at 14,000×g for 5 minutes. The supernatant was extracted and analyzed for metal content. For Mn2+ and Mg2+, the relative concentrations were measured both before and after adding 5 molar equivalents of MnCl2 to the buffer-exchanged enzyme. While structures of as-purified FolE2 from N. gonorrhoeae were modeled with Zn2+ based on X-ray fluorescence from the crystals, ICP-MS data suggest this is not the correct assignment for crystals of the as-purified enzyme from B. pseudomallei. Mg2+, which has also been demonstrated to support catalytic activity, was identified as the dominant divalent metal ion among those tested. No other divalent metals tested approached an equimolar ratio with enzyme. Following incubation with MnCl2, ICP-MS data support a 1:1 molar ratio of Mn2+ to enzyme, suggesting Mn2+ was bound in the active site, in agreement with previous crystallographic studies that followed a similar protocol. However, the ICP-MS detected concentration of Mg2+ was unchanged following incubation. This result, together with known strict octahedral coordination of Mg2+ and a Mg-O bond length that is substantially longer (˜2.38 Å) than the ideal (˜2.1 Å)12, led us to conclude that the metal binding site of the as-purified enzyme was occupied by Na+. Sodium is present at high concentrations in both the storage buffer and crystallization condition, can have lower coordination numbers, and has an ideal Na—O bond length of ˜2.4 Å.

A number of implementations have been described. Nevertheless, it will be understood that various modifications may be made without departing from the spirit and scope of the disclosure. Accordingly, other implementations are within the scope of the following claims.

1. A system for treating melioidosis, comprising:
a first antimicrobial agent comprising trimethoprim at a subinhibitory concentration; and
a second antimicrobial agent comprising a FolE2 enzyme inhibitor selected from dehydrocostus lactone, parthenolide, or β-lapachone;
wherein the combination of the first antimicrobial agent and the second antimicrobial agent exhibits chemical synthetic lethality against Burkholderia pseudomallei while having minimal effect on commensal bacteria.

2. The system of claim 1, wherein the subinhibitory concentration of trimethoprim is between 5 μM and 30 μM.

3. The system of claim 2, wherein the subinhibitory concentration of trimethoprim is between 5 μM and 30 μM.

4. The system of claim 1, wherein the FolE2 enzyme inhibitor is dehydrocostus lactone.

5. The system of claim 4, wherein the dehydrocostus lactone has an IC₅₀ value of between 1.7 μM and 2.5 μM in the presence of trimethoprim.

6. The system of claim 1, wherein the FolE2 enzyme inhibitor is β-lapachone.

7. The system of claim 6, wherein the β-lapachone has an IC₅₀ value of between 3.2 μM and 6.4 μM in the presence of trimethoprim.

8. The system of claim 1, wherein the FolE2 enzyme inhibitor is parthenolide.

9. The system of claim 1, wherein the combination exhibits greater than 90% growth suppression against Burkholderia pseudomallei.

10. The system of claim 1, wherein the FolE2 enzyme inhibitor acts as a mechanism-based inhibitor that covalently modifies a catalytic cysteine residue of the FolE2 enzyme.

11. The system of claim 10, wherein the catalytic cysteine residue is Cys154.

12. A method for treating melioidosis in a subject, comprising:
administering to the subject a therapeutically effective amount of a pharmaceutical composition comprising trimethoprim at a subinhibitory concentration and a FolE2 enzyme inhibitor;
wherein the FolE2 enzyme inhibitor is selected from dehydrocostus lactone, parthenolide, or β-lapachone; and
wherein the combination exhibits chemical synthetic lethality against Burkholderia pseudomallei.

13. The method of claim 12, wherein the subinhibitory concentration of trimethoprim is between 5 μM and 30 μM.

14. The method of claim 13, wherein the subinhibitory concentration of trimethoprim is about 20 μM.

15. The method of claim 12, wherein the FolE2 enzyme inhibitor is dehydrocostus lactone having an IC₅₀ value of between 1.7 μM and 2.5 μM in the presence of trimethoprim.

16. The method of claim 12, wherein the FolE2 enzyme inhibitor acts as a mechanism-based inhibitor that covalently modifies a catalytic cysteine residue of the FolE2 enzyme.

17. The method of claim 16, wherein the catalytic cysteine residue is Cys154.

18. The method of claim 12, wherein the combination exhibits greater than 90% growth suppression against Burkholderia pseudomallei while having minimal effect on commensal bacteria selected from Bacteroides fragilis, Bifidobacterium longum, Clostridium sporogenes, and Parabacteroides distasonis.

19. A method for identifying antimicrobial drug targets, comprising:
screening a library of compounds for growth inhibition of a target bacterial pathogen in the presence of a subinhibitory concentration of a known antibiotic;
identifying compounds that exhibit no growth inhibition individually but cause growth inhibition when combined with the subinhibitory concentration of the known antibiotic;
determining the molecular target of the identified compounds; and
validating the molecular target as a conditionally essential enzyme that becomes essential in the presence of the subinhibitory concentration of the known antibiotic.

20. The method of claim 19, wherein the library of compounds comprises natural products and FDA-approved small-molecule drugs.

21. The method of claim 19, wherein growth inhibition is measured by optical density at 600 nm after incubation for 24 to 48 hours.

22. The method of claim 19, wherein the identified compounds are mechanism-based inhibitors that covalently modify a catalytic amino acid residue.

23. The method of claim 22, wherein the mechanism-based inhibitors comprise sesquiterpene lactones with exocyclic enone moieties.
